(19) United States
(12) Patent Application Publication
(10) Pub. No.: US 2001/0025098 A1
(43) Pub. Date: Sep. 27, 2001

(54) Human membrane-spanning proteins

(75) Inventors: Y. Tom Tang, San Jose, CA (US); Olga Bandman, Mountain View, CA (US); Preeti Lal, Santa Clara, CA (US); Jennifer L. Hillman, Mountain View, CA (US); Henry Yue, Sunnyvale, CA (US); Neil C. Corley, Mountain View, CA (US); Karl J. Guegler, Menlo Park, CA (US); Matthew R. Kaser, Castro Valley, CA (US); Mariah R. Baughn, San leandro, CA (US); Purvi Shah, Sunnyvale, CA (US)

Correspondence Address: INCYTE GENOMICS, INC., PATENT DEPARTMENT, 3160 Porter Drive, Palo Alto, CA, 94304, US

(73) Assignee: Incyte Pharmaceuticals, Inc.

(21) Appl. No.: 09/823,356

(22) Filed: Mar. 30, 2001

Related U.S. Application Data
(63) Continuation of application No. 09/039,307, filed on Mar. 13, 1998.

(51) Int. Cl. C12Q 1/68; C07H 21/04; C12P 21/02; C07K 14/705; C07K 16/18

(52) U.S. Cl. 530/350; 435/6; 435/69.1; 435/325; 536/23.5; 530/388.22

(57) ABSTRACT

The invention provides a human membrane spanning proteins (MSPs) and polynucleotides which identify and encode MSPs. The invention also provides expression vectors, host cells, antibodies, agonists, and antagonists. The invention also provides methods for treating or preventing disorders associated with expression of MSPs.

This application is a CONTINUATION application of U.S. application Ser. No. 09/039,307, filed on Mar. 13, 1998, originally entitled HUMAN MEMBRANE SPANNING PROTEINS, which is hereby expressly incorporated by reference.

### FIELD OF THE INVENTION

This invention relates to nucleic acid and amino acid sequences of human membrane spanning proteins and to the use of these sequences in the diagnosis, treatment, and prevention of neoplastic, immunological, and reproductive disorders.

### BACKGROUND OF THE INVENTION

Eukaryotic organisms are distinct from prokaryotes in possessing many intracellular organelle and vesicle structures. Many of the metabolic reactions which distinguish eukaryotic biochemistry from prokaryotic biochemistry take place within these structures. In particular, many cellular functions require very strict reagent conditions, and the organelles and vesicles enable compartmentalization and isolation of reactions which might otherwise cripple cytosolic metabolic processes. The organelles are bounded by single or double membranes each made up of lipid bilayer sheets and include mitochondria; smooth and rough endoplasmic reticuli; sarcoplasmic reticulum; and the Golgi body. The lipid bilayer sheets are composed of phosphoglycerides, fatty acids, cholesterol, glycolipids, proteoglycans, and proteins. The vesicles are bounded by single membranes and include phagosomes; lysosomes; endosomes; peroxisomes; and secretory vesicles.

Biological membranes are highly selective permeability barriers that contain ion pumps, gates, and specific receptors for external stimuli which transmit biochemical signals across the membranes. These membranes also contain proteins which interact with these pumps, gates, and receptors to amplify and regulate transmision of these signals.

Plasma Membrane Proteins

Plasma membrane proteins (MPs) are divided into two groups based upon the mechanism of protein removal from the membrane. Extrinsic or peripheral membrane proteins can be removed using extremes of ionic strength or pH, urea, or other disruptors of protein interactions. Intrinsic or integral membrane proteins are released only when the lipid bilayer of the membrane is dissolved by detergent.

The majority of known integral membrane proteins are transmembrane proteins which are characterized by an extracellular, a transmembrane, and an intracellular domain. Transmembrane proteins are typically embedded in the cell membrane by one or more regions comprising 15 to 25 hydrophobic amino acids which are predicted to adopt an α-helical conformation. Transmembrane proteins are classified as bitopic (Types I and II) and polytopic (Types III and IV) (Singer, S. J. (1990) Annu. Rev. Cell Biol. 6:247-296). Bitopic proteins span the membrane once while polytopic proteins contain multiple membrane-spanning segments. Type III integral membrane proteins have multiple transmembrane stretches of hydrophobic residues. Transmembrane proteins carry out a variety of important cellular functions such as acting as cell-surface receptor proteins involved in signal transduction, e.g., growth factor and differentiation factor receptors and receptor-interacting proteins; transport of ions or metabolites, e.g., gap junction channels (connexins), and ion channels; cell anchoring proteins, extracellular matrix (ECM)-binding proteins, lectins, integrins, and fibronectins; or cell recognition molecules, e.g., cluster of differentiation (CD) antigens, glycoproteins and mucins.

Many MPs contain amino acid sequence motifs that serve to localize proteins to specific subcellular sites. Examples of these motifs include, e.g., PDZ domains, KDEL, RGD, NGR, GSL, von Willebrand factor A (vWFA) domains, and EGF-like domains. Furthermore, MPs may also contain amino acid sequence motifs that serve to interact with extracellular or intracellular molecules, such as carbohydrate recognition domains (CRD). RGD, NGR, GSL motif-containing peptides have been used as drug delivery agents in targeted cancer treatment of tumor vasculature (Arap, W. et al. (1998) Science 279:377-380).

Chemical modification of amino acid residue side chains alters the manner in which a protein interacts with other molecules, for example, phospholipid membranes. Examples of such chemical modifications to amino acid residue side chains are covalent bond formiation with glycosaminoglycans, oligosaccharides, phospholipids, acetyl and palmitoyl moieties, ADP-ribose, phosphate, and sulphate groups.

Premessenger RNA encoding membrane proteins may have alternative splice sites which give rise to proteins encoded by the same gene but with different messenger RNA and amino acid sequences. Splice variant membrane proteins may interact with other ligand and protein isoforms.

Tetraspan Family Proteins

The transmembrane 4 superfamily (TM4SF) or tetraspan family are a multigene family encoding type III integral membrane proteins (Wright, M. D. and Tomlinson, M. G. (1994) Immunol. Today 15:588). The TM4SF family comprises a superfamily of membrane proteins which traverse the cell membrane four times. Members of the TM4SF include a number of platelet and endothelial cell membrane proteins, the platelet and melanoma-associated antigens, leukocyte surface glycoproteins, the colonal carcinoma antigens, the tumor-associated antigens, and surface proteins of the schistosome parasites (Jankowski, S. A. (1994) Oncogene 9:1205-1211). The members of the TM4SF share about 25-30% amino acid sequence identity with one another.

A number of TM4SF members have been implicated in signal transduction, control of cell adhesion, regulation of cell growth and proliferation, including development and oncogenesis, and motility, including the ability to suppress metastatic potential. Expression of a number of TM4SF proteins is associated with a variety of tumors and the level of expression may be altered when cells are growing or activated.

Proton Pumps

Proton ATPases are a large class of membrane-proteins that use the energy of ATP hydrolysis to generate an electrochemical proton gradient across a membrane. The resultant gradient may be used to transport other ions across the membrane (Na⁺, K⁺, or Cl⁻) or to maintain an acidic environment important to the function of many cellular vesicles (Mellman, I. et al. (1986) Annu. Rev. Biochem. 55:663-700). Proton ATPases are further subdivided into the mitochondrial F-ATPases, the plasma membrane ATPases, and the vacuolar ATPases. The vacuolar ATPases establish and maintain an acidic pH within various vesicles involved in the processes of endocytosis and exocytosis.

Proton-coupled, 12 membrane-spanning domain transporters such as PEPT 1 and PEPT 2 are responsible for gastrointestinal absorption and for renal reabsorbtion of peptides using an electrochemical H⁺ gradient as the driving force. A heterodimeric peptide transporter, consisting of TAP 1 and TAP 2, is associated with antigen processing. Peptide antigens are transported across the membrane of the endoplasmic reticulum so they can be presented to the major histocompatibility complex class I molecules. Each TAP protein consists of multiple hydrophobic membrane spanning segments and a highly conserved ATP-binding cassette (Boll, M. et al. (1996) Proc. Natl. Acad. Sci. USA 93:284-289). Pathogenic microorganisms, such as herpes simplex virus, may encode inhibiotrs of TAP-mediated peptide transport in order to evade immune surveillance (Marusina, K. and Manaco, J. J. (1996) Curr. Opin. Hematol. 3:19-26).

Scavenger Receptors

Macrophage scavenger receptors with broad ligand specificity have been suggested to take part in the binding of low density lipoproteins (LDL) and foreign antigens. Scavenger receptors types I and II are trimeric membrane proteins with a small N-terminal intracellular domain, a transmembrane domain, a large extracellular domain, and a C-terminal cysteine-rich domain. The extracellular domain contains a short spacer domain, an α-helical coiled-coil domain, and a triple helical collagenous domain. These receptors have been shown to bind a spectrum of ligands, including chemically modified lipoproteins and albumin, polyribonucleotides, polysaccharides, phospholipids, and asbestos (Matsumoto, A. et al. (1990) Proc. Natl. Acad. Sci. USA 87:9133-9137; and Elomaa, O. et al. (1995) Cell 80:603-609).

The scavenger receptors are thought to play a key role in atherogenesis by mediating uptake of modified LDL in arterial walls, and in host defense by binding bacterial endotoxins, bacteria, and protozoa.

Ion Channels

Ion channels are found in the plasma membranes of virtually every cell in the body. For example, chloride channels mediate a variety of cellular functions including regulation of membrane potentials and absorption and secretion of ions across epithelial membranes. When present in intracellular membranes of the Golgi apparatus and endocytic vesicles, chloride channels also regulate organelle pH (see, e.g., Greger, R. (1988) Annu. Rev. Physiol. 50:111-122). Electrophysiological and pharmacological measurements including ion conductance, current-voltage relationships, and sensitivity to modulators suggest that different chloride channels exist in muscles, neurons, fibroblasts, epithelial cells, and lymphocytes.

Many of the channels have sites for phosphorylation by one or more protein kinases including protein kinase A, protein kinase C, tyrosine kinase, and casein kinase II, all of which regulate ion channel activity in cells. Inapproriate phosphorylation of proteins in cells has been linked to changes in cell cycle events and differentiation status. Changes in the cell cycle have been linked to induction of apoptosis or cancer. Changes in differentiation status of the cell have been linked to, for example, immune diseases and disorders, diseases and disorders of skeletal muscle, and diseases and disorders of the reproductive system.

G-Protein Coupled Receptors

G-protein coupled receptors (GPCR) are a superfamily of integral membrane proteins which transduce extracellular signals. GPCRs include receptors for biogenic amines; for lipid mediators of inflammation, peptide hormones, and sensory signal mediators.

The structure of these highly-conserved receptors consists of seven hydrophobic transmembrane (serpentine) regions, cysteine disulfide bridges between the second and third extracellular loops, an extracellular N-terminus, and a cytoplasmic C-terminus. Three extracellular loops alternate with three intracellular loops to link the seven transmembrane regions. The most conserved parts of these proteins are the transmembrane regions and the first two cytoplasmic loops. A conserved, acidic-Arg-aromatic residue triplet present in the second cytoplasmic loop may interact with the G proteins. The consensus pattern of the G-protein coupled receptors signature (PS00237; SWISSPROT) is characteristic of most proteins belonging to this superfamily (Watson, S. and S. Arkinstall (1994) The G-protein Linked Receptor Facts Book, Academic Press, San Diego Calif., pp. 2-6).

Mutations and changes in transcriptional activation of G protein-encoding genes have been associated with, for example, schizophrenia, Parkinson's disease, Alzheimer's disease, drug addiction, and feeding disorders.

ABC Transporters

The ATP-binding cassette (ABC) transporters, also called the “traffic ATPases”, comprise a superfamily of membrane proteins that mediate transport and channel functions in prokaryotes and eukaryotes (Higgins, C. F. (1992) Annu. Rev. Cell Biol. 8:67-113). ABC proteins share a similar overall structure and significant sequence homology. All ABC proteins contain a conserved domain of approximately two hundred amino acid residues which includes one or more nucleotide binding domains. Mutations in ABC transporter genes are associated with, for example, hyperbilirubinemia II/Dubin-Johnson syndrome, recessive Stargardt's disease, X-linked adrenoluekodystrophy, multidrug resistance, celiac disease, and cystic fibrosis.

Tumor Antigens

Tumor antigens are surface molecules that are differentially expressed in tumor cells relative to non-tumor tissues. Tumor antigens distinguish tumor cells immunologically from normal cells and provide diagnostic and therapeutic targets for human cancers (Takagi, S. et al. (1995) Int. J. Cancer 61:706-715; Liu, E. et al. (1992) Oncogene 7:1027-1032).

Mitochondrial Membrane Proteins

The mitochondrial electron transport (or respiratory) chain is a series of three enzyme complexes in the mitochondrial membrane that is responsible for the transport of electrons from NADH to oxygen and the coupling of this oxidation to the synthesis of ATP (oxidative phosphorylation). ATP then provides the primary source of energy for driving the many energy-requiring reactions of a cell.

Most of the protein components of the mitochondrial respiratory chain are the products of nuclear encoded genes that are imported into the mitochondria, and the remainder are products of mitochondrial genes. Defects and altered expression of enzymes in the respiratory chain are associated with a variety of disease conditions in man, including, for example, neurodegenerative diseases, myopathies, and cancer.

Endopolasmic Reticulum Membrane Proteins

The normal functioning of the eukaryotic cell requires that all newly synthesized proteins be correctly folded, modified, and delivered to specific intra- and extracellular sites. Newly synthesized membrane and secretory proteins enter a cellular sorting and distribution network during or immediately after synthesis (cotranslationally or posttranslationally) and are routed to specific locations inside and outside of the cell. The initial compartment in this process is the endoplasmic reticulum (ER) where proteins undergo modifications such as glycosylation, disulfide bond formation, and assembly into oligomers. The proteins are then transported through an additional series of membrane-bound compartments which include the various cisternae of the Golgi complex, where further carbohydrate modifications occur. Transport between compartments occurs by means of vesicles that bud and fuse in a specific manner. Once within the secretory pathway, proteins do not have to cross a membrane to reach the cell surface.

The majority of proteins processed through the ER are transported out of the organelle however some are retained. The signal for retention in the ER in mammalian cells consists of the tetrapeptide sequence, KDEL, located at the carboxy terminus of proteins (Munro, S. (1986) Cell 46:291-300). Proteins containing this sequence leave the ER but are quickly retrieved from the early Golgi compartment and returned to the ER, while proteins without this signal continue through the distribution pathway.

Disruptions in the cellular secretory pathway have been implicated in several human diseases. In familial hypercholesterolemia the low density lipoprotein receptors remain in the ER, rather than moving to the cell surface (Pathak, R. K. (1988) J. Cell Biol. 106:1831-1841).

Presenilins are localized to the ER and may regulate cellular calcium homeostasis in early-onset Alzheimer's disease and in Down syndrome. Changes in ER-derived calcium homeostasis have also been associated with, for example, cardiomyopathy, cardiac hypertrophy, myotonic dystrophy, Brody disease, Smith-McCort dysplasia, and diabetes mellitus.

Intercellular Communication Membrane Proteins

Intercellular communication is essential for the development and survival of multicellular organisms. Cells communicate with one another through the secretion and uptake of protein signaling molecules. The uptake of proteins into the cell is achieved by endocytosis, in which the interaction of signaling molecules with the plasma membrane surface, often via binding to specific receptors, results in the formation of plasma membrane-derived vesicles that enclose and transport the molecules into the cytosol. The secretion of proteins from the cell is achieved by exocytosis, in which molecules inside of the cell are packaged into membrane-bound transport vesicles derived from the trans Golgi network. These vesicles fuse with the plasma membrane and release their contents into the surrounding extracellular space. Endocytosis and exocytosis result in the removal and addition of plasma membrane components, and the recycling of these components is essential to maintain the integrity, identity, and functionality of both the plasma membrane and internal membrane-bound compartments.

Endocytosis involves the internalizing of nutrients, solutes or small particles (pinocytosis); or large particles such as internalized receptors, viruses, bacteria, or bacterial toxins (phagocytosis). In exocytosis, molecules are transported to the cell surface. Exocytosis facilitates the placement or localization of membrane-bound receptors or other membrane proteins and the secretion of hormones, neurotransmitters, digestive enzymes, and wastes. Endocytosis and exocytosis are fundamental to the function of all types of cells.

Isolation of intracellular organelles from rat liver has demonstrated the presence of two distinct organelles, the lysosome and the peroxisome (de Duve, C. (1996) Ann. N.Y. Acad. Sci. 804:1-10). Lysosomes are the site of degradation of obsolete intracellular material during autophagy and of extracellular molecules following endocytosis and phagocytosis. They are derived from endosomes, which in turn are formed from budding of the trans-Golgi network or from clathrin-coated membrane vesicles invaginating from the plasma membrane.

Protein sorting by transport vesicles, such as the endosome, has important consequences for a variety of physiological processes including cell surface growth, the biogenesis of distinct intracellular organelles, endocytosis, and the controlled release of hormones and neurotransmitters (Rothman, J. E. and Wieland, F. T. (1996) Science 272:227-234). In particular, neurodegenerative disorders and other neuronal pathologies are associated with biochemical flaws during endosomal protein sorting or endosomal biogenesis (Mayer R. J. et al. (1996) Adv. Exp. Med. Biol. 389:261-269).

Peroxisomes are independent organelles and are not members of the secretory pathway family of organelles. They are characterized by a single membrane and a finely granulated matrix and are the site of many peroxide-generating oxidative reactions in the cell. Peroxisomes are unique among eukaryotic organelles in that their size, number, and enzyme content vary depending upon organism, cell type, and metabolic needs. The majority of peroxisome-associated proteins are membrane-bound or are found proximal to the cytosolic or the lumenal side of the peroxisome membrane (Waterham, H. R. and Cregg, J. M. (1996) BioEssays 19:57-66).

Genetic defects in peroxisome proteins which result in peroxisomal deficiencies have been linked to a number of human pathologies, including Zellweger syndrome, rhizomelic chonrodysplasia punctata, X-linked adrenoleukodystrophy, acyl-CoA oxidase deficiency, bifunctional enzyme deficiency, classical Refsum's disease, DHAP alkyl transferase deficiency, and acatalasemia (Moser, H. W. and Moser, A. B. (1996) Ann. NY Acad. Sci. 804:427-441). In addition, Gartner, J. et al. (1991; Pediatr. Res. 29:141-146) found a 22 kDa integral membrane protein associated with lower density peroxisome-like subcellular fractions in patients with Zellweger syndrome.

Normal embryonic development and control of germ cell maturation is modulated by a number of secretory proteins which interact with their respective membrane-bound receptor. Cell fate during embryonic development is determined, for example, by members of the activin/TGF-β superfamily, cadherin(s), IGF-2, and other morphogen(s). In addition, proliferation, maturation, and redifferentiation of germ cell and reproductive tissues are regulated, for example, by IGF-2, inhibin(s), activin(s), and follistatin(s) (Petraglia, F. (1997) Placenta 18:3-8; and Mather, J. P. et al. (1997) Proc. Soc. Exp. Biol. Med. 215:209-222).

Lymphocyte and Leukocyte Membrane Proteins

The B-cell response to antigens, which is modulated through receptors, is an essential component of the normal immune system. Mature B cells recognize foreign antigens through B cell receptors (BCR) and produce specific antibodies which bind the foreign antigens. The antigen/receptor complex is internalized, and the antigen is proteolytically processed. To generate an efficient response to complex antigens, the assistance of the BCR, BCR associated proteins, and T cell is required. A small part of the antigen remains complexed with major histocompatability complex-II (MHCII) molecules on the surface of the B cells where the complex can be recognized by T cells. MHCI molecules are on the surface of non-lymphoid tissue and present antigens to macrophages or other lymphoid cell types. T cells recognize and are activated by the MHCI-antigen complex through interactions with the T cell receptor/CD3 complex, a T cell surface multimeric, multiphenotypic protein located in the plasma membrane. T cells activated by antigen presentation secrete a variety of lymphokines that induce B cell maturation, activate macrophages, and kill target cells.

Leukocytes have a fundamental role in the inflammatory and immune resonse, and include monocytes/macrophages, mast cells, polymorphonucleoleukocytes, natural killer cells, nuetrophils, eosinophils, basophils, and myeloid precursors. Leukocyte membrane proteins include members of the CD antigens, N-CAM, I-CAM, human leukocyte antigen (HLA) class I and HLA class II gene products, immunoglobulins, immunoglobulin receptors, complement, complement receptors, interferons, interferon receptors, interleukin receptors, and chemokine receptors.

Abnormal lymphocyte and leukocyte activity has been associated with, for example, AIDS, immune hypersensitivity, leukemias, leukopenia, systemic lupus, granulomatous disease, and eosinophilia.

Apoptosis

A variety of ligands and their cellular receptors, enzymes, tumor suppressors, viral gene products, pharmacological agents, and inorganic ions have important positive or negative roles in regulating and implementing the apoptotic destruction of a cell. Although some specific components of the apoptotic pathway have been identified and characterized, many interactions between the proteins involved are undefined, leaving major aspects of the pathway unknown.

A requirement for calcium in apoptosis was previously suggested by studies showing the involvement of calcium levels in DNA cleavage and Fas-mediated cell death (Hewish, D. R. and L. A. Burgoyne (1973) Biochem. Biophys. Res. Commun. 52:504-510; Vignaux, F. et al. (1995) J. Exp. Med. 181:781-786; and Oshimi, Y. and S. Miyazaki (1995) J. Immunol. 154:599-609). Other studies show that intracellular calcium concentrations increase when apoptosis is triggered in thymocytes by either T cell receptor cross-linking or by glucocorticoids, and cell death can be prevented by blocking this increase (McConkey, D. J. et al. (1989) J. Immunol. 143:1801-1806; and McConkey, D. J. et al. (1989) Arch. Biochem. Biophys. 269:365-370).

The discovery of new human membrane spanning proteins and the polynucleotides encoding it satisfies a need in the art by providing new compositions which are useful in the diagnosis, treatment, and prevention of neoplastic, immunological, and reproductive disorders.

### SUMMARY OF THE INVENTION

The invention features a substantially purified polypeptide, human membrane spanning proteins (MSPs), having an amino acid sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, and SEQ ID NO:17 (SEQ ID NO:1 through SEQ ID NO:17), or fragments thereof.

The invention further provides a substantially purified variant having at least 90% amino acid identity to the amino acid sequences of SEQ ID NO:1 through SEQ ID NO:17, or to a fragment of any of these sequences. The invention also provides an isolated and purified polynucleotide encoding the polypeptide comprising an amino acid sequence selected from the group consisting of SEQ ID NO:1 through SEQ ID NO:17, or fragments thereof. The invention also includes an isolated and purified polynucleotide variant having at least 90% polynucleotide seqeunce identity to the polynucleotide encoding the polypeptide comprising an amino acid sequence selected from the group consisting of SEQ ID NO:1 through SEQ ID NO:17, or fragments thereof.

Additionally, the invention provides an isolated and purified polynucleotide which hybridizes under stringent conditions to the polynucleotide encoding the polypeptide comprising an amino acid sequence selected from the group consisting of SEQ ID NO:1 through SEQ ID NO:17, or fragments thereof, as well as an isolated and purified polynucleotide having a sequence which is complementary to the polynucleotide encoding the polypeptide comprising the amino acid sequence selected from the group consisting of SEQ ID NO:1 through SEQ ID NO:17, or fragments thereof.

The invention also provides an isolated and purified polynucleotide comprising a polynucleotide sequence selected from the group consisting of SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, and SEQ ID NO:34 (SEQ ID NO:18 through SEQ ID NO:34), or fragments thereof. The invention further provides an isolated and purified polynucleotide variant having at least 90% polynucleotide sequence identity to the polynucleotide sequence comprising a polynucleotide sequence selected from the group consisting of SEQ ID NO:18 through SEQ ID NO:34, or fragment thereof, as well as an isolated and purified polynucleotide having a sequence which is complementary to the polynucleotide comprising a polynucleotide sequence selected from the group consisting of SEQ ID NO:18 through SEQ ID NO:34, or fragments thereof.

The invention further provides an expression vector containing at least a fragment of the polynucleotide encoding the polypeptide comprising an amino acid sequence selected from the group consisting of SEQ ID NO:1 through SEQ ID NO:17, or fragments thereof. In another aspect, the expression vector is contained within a host cell.

The invention also provides a method for producing a polypeptide comprising the amino acid sequence selected from the group consisting of SEQ ID NO:1 through SEQ ID NO:17, or fragments thereof, the method comprising the steps of: (a) culturing the host cell containing an expression vector containing at least a fragment of a polynucleotide encoding the polypeptide under conditions suitable for the expression of the polypeptide; and (b) recovering the polypeptide from the host cell culture.

The invention also provides a pharmaceutical composition comprising a substantially purified polypeptide having the amino acid sequence selected from the group consisting of SEQ ID NO:1 through SEQ ID NO:17, or fragments thereof in conjunction with a suitable pharmaceutical carrier.

The invention further includes a purified antibody which binds to a polypeptide comprising the amino acid sequence selected from the group consisting of SEQ ID NO:1 through SEQ ID NO:17, or fragments thereof, as well as a purified agonist and a purified antagonist to the polypeptide.

The invention also provides a method for treating or preventing a neoplastic disorder, the method comprising administering to a subject in need of such treatment an effective amount of an antagonist of the polypeptide having an amino acid sequence selected from the group consisting of SEQ ID NO:1 through SEQ ID NO:17, or fragments thereof.

The invention also provides a method for treating or preventing an immunological disorder, the method comprising administering to a subject in need of such treatment an effective amount of an antagonist of the polypeptide having an amino acid sequence selected from the group consisting of SEQ ID NO:1 through SEQ ID NO:17, or fragments thereof.

The invention also provides a method for treating or preventing a reproductive disorder, the method comprising administering to a subject in need of such treatment an effective amount of an antagonist of the polypeptide having an amino acid sequence selected from the group consisting of SEQ ID NO:1 through SEQ ID NO:17, or fragments thereof.

The invention also provides a method for detecting a polynucleotide encoding the polypeptide comprising the amino acid sequence selected from the group consisting of SEQ ID NO:1 through SEQ ID NO:17, or fragments thereof in a biological sample containing nucleic acids, the method comprising the steps of: (a) hybridizing the complement of the polynucleotide sequence encoding the polypeptide comprising the amino acid sequence selected from the group consisting of SEQ ID NO:1 through SEQ ID NO:17, or fragments thereof to at least one of the nucleic acids of the biological sample, thereby forming a hybridization complex; and (b) detecting the hybridization complex, wherein the presence of the hybridization complex correlates with the presence of a polynucleotide encoding the polypeptide in the biological sample. In one aspect, the nucleic acids of the biological sample are amplified by the polymerase chain reaction prior to the hybridizing step.

### DESCRIPTION OF THE INVENTION

Before the present proteins, nucleotide sequences, and methods are described, it is understood that this invention is not limited to the particular methodology, protocols, cell lines, vectors, and reagents described, as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a,” “an,” and “the” include plural reference unless the context clearly dictates otherwise. Thus, for example, a reference to “a host cell” includes a plurality of such host cells, and a reference to “an antibody” is a reference to one or more antibodies and equivalents thereof known to those skilled in the art, and so forth.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices, and materials are now described. All publications mentioned herein are cited for the purpose of describing and disclosing the cell lines, vectors, and methodologies which are reported in the publications and which might be used in connection with the invention. Nothing herein is to be construed as an admission that the invention is not entitled to antedate such disclosure by virtue of prior invention.

DEFINITIONS

“MSP,” as used herein, refers to the amino acid sequences of substantially purified MSP obtained from any species, particularly a mammalian species, including bovine, ovine, porcine, murine, equine, and preferably the human species, from any source, whether natural, synthetic, semi-synthetic, or recombinant.

The term “agonist,” as used herein, refers to a molecule which, when bound to MSP, increases or prolongs the duration of the effect of MSP. Agonists may include proteins, nucleic acids, carbohydrates, or any other molecules which bind to and modulate the effect of MSP.

An “allele” or an “allelic sequence,” as these terms are used herein, is an alternative form of the gene encoding MSP. Alleles may result from at least one mutation in the nucleic acid sequence and may result in altered mRNAs or in polypeptides whose structure or function may or may not be altered. Any given natural or recombinant gene may have none, one, or many allelic forms. Common mutational changes which give rise to alleles are generally ascribed to natural deletions, additions, or substitutions of nucleotides. Each of these types of changes may occur alone, or in combination with the others, one or more times in a given sequence.

“Altered” nucleic acid sequences encoding MSP, as described herein, include those sequences with deletions, insertions, or substitutions of different nucleotides, resulting in a polynucleotide the same MSP or a polypeptide with at least one functional characteristic of MSP. Included within this definition are polymorphisms which may or may not be readily detectable using a particular oligonucleotide probe of the polynucleotide encoding MSP, and improper or unexpected hybridization to alleles, with a locus other than the normal chromosomal locus for the polynucleotide sequence encoding MSP. The encoded protein may also be “altered,” and may contain deletions, insertions, or substitutions of amino acid residues which produce a silent change and result in a functionally equivalent MSP. Deliberate amino acid substitutions may be made on the basis of similarity in polarity, charge, solubility, hydrophobicity, hydrophilicity, and/or the amphipathic nature of the residues, as long as the biological or immunological activity of MSP is retained. For example, negatively charged amino acids may include aspartic acid and glutamic acid, positively charged amino acids may include lysine and arginine, and amino acids with uncharged polar head groups having similar hydrophilicity values may include leucine, isoleucine, and valine; glycine and alanine; asparagine and glutamine; serine and threonine; and phenylalanine and tyrosine.

The terms “amino acid” or “amino acid sequence,” as used herein, refer to an oligopeptide, peptide, polypeptide, or protein sequence, or a fragment of any of these, and to naturally occurring or synthetic molecules. In this context, “fragments”, “immunogenic fragments”, or “antigenic fragments” refer to fragments of MSP which are preferably about 5 to about 15 amino acids in length and which retain some biological activity or immunological activity of MSP. Where “amino acid sequence” is recited herein to refer to an amino acid sequence of a naturally occurring protein molecule, “amino acid sequence” and like terms are not meant to limit the amino acid sequence to the complete native amino acid sequence associated with the recited protein molecule.

“Amplification,” as used herein, relates to the production of additional copies of a nucleic acid sequence. Amplification is generally carried out using polymerase chain reaction (PCR) technologies well known in the art (See, e.g., Dieffenbach, C. W. and G. S. Dveksler (1995) PCR Primer, a Laboratory Manual, Cold Spring Harbor Press, Plainview N.Y., pp.1-5.)

The term “antagonist,” as it is used herein, refers to a molecule which, when bound to MSP, decreases the amount or the duration of the effect of the biological or immunological activity of MSP. Antagonists may include proteins, nucleic acids, carbohydrates, antibodies, or any other molecules which decrease the effect of MSP.

As used herein, the term “antibody” refers to intact molecules as well as to fragments thereof, such as Fa, F(ab′)₂, and Fv fragments, which are capable of binding the epitopic determinant. Antibodies that bind MSP polypeptides can be prepared using intact polypeptides or using fragments containing small peptides of interest as the immunizing antigen. The polypeptide or oligopeptide used to immunize an animal (e.g., a mouse, a rat, or a rabbit) can be derived from the translation of RNA, or synthesized chemically, and can be conjugated to a carrier protein if desired. Commonly used carriers that are chemically coupled to peptides include bovine serum albumin, thyroglobulin, and keyhole limpet hemocyanin (KLH). The coupled peptide is then used to immunize the animal.

The term “antigenic determinant,” as used herein, refers to that fragment of a molecule (i.e., an epitope) that makes contact with a particular antibody. When a protein or a fragment of a protein is used to immunize a host animal, numerous regions of the protein may induce the production of antibodies which bind specifically to antigenic determinants (given regions or three-dimensional structures on the protein). An antigenic determinant may compete with the intact antigen (i.e., the immunogen used to elicit the immune response) for binding to an antibody.

The term “antisense,” as used herein, refers to any composition containing a nucleic acid sequence which is complementary to a specific nucleic acid sequence. The term “antisense strand” is used in reference to a nucleic acid strand that is complementary to the “sense” strand. Antisense molecules may be produced by any method including synthesis or transcription. Once introduced into a cell, the complementary nucleotides combine with natural sequences produced by the cell to form duplexes and to block either transcription or translation. The designation “negative” can refer to the antisense strand, and the designation “positive” can refer to the sense strand.

As used herein, the term “biologically active,” refers to a protein having structural, regulatory, or biochemical functions of a naturally occurring molecule. Likewise, “immunologically active” refers to the capability of the natural, recombinant, or synthetic MSP, or of any oligopeptide thereof, to induce a specific immune response in appropriate animals or cells and to bind with specific antibodies.

The terms “complementary” or “complementarity,” as used herein, refer to the natural binding of polynucleotides under permissive salt and temperature conditions by base pairing. For example, the sequence “A-G-T” binds to the complementary sequence “T-C-A.” Complementarity between two single-stranded molecules may be “partial,” such that only some of the nucleic acids bind, or it may be “complete,” such that total complementarity exists between the single stranded molecules. The degree of complementarity between nucleic acid strands has significant effects on the efficiency and strength of the hybridization between the nucleic acid strands. This is of particular importance in amplification reactions, which depend upon binding between nucleic acids strands, and in the design and use of peptide nucleic acid (PNA) molecules.

A “composition comprising a given polynucleotide sequence” or a “composition comprising a given amino acid sequence,” as these terms are used herein, refer broadly to any composition containing the given polynucleotide or amino acid sequence. The composition may comprise a dry formulation, an aqueous solution, or a sterile composition. Compositions comprising polynucleotide sequences encoding MSP or fragments of MSP may be employed as hybridization probes. The probes may be stored in freeze-dried form and may be associated with a stabilizing agent such as a carbohydrate. In hybridizations, the probe may be deployed in an aqueous solution containing salts (e.g., NaCl), detergents (e.g., SDS), and other components (e.g., Denhardt's solution, dry milk, salmon sperm DNA, etc.).

The phrase “consensus sequence,” as used herein, refers to a nucleic acid sequence which has been resequenced to resolve uncalled bases, extended using XL-PCR (Perkin Elmer, Norwalk, Conn.) in the 5′ and/or the 3′ direction, and resequenced, or which has been assembled from the overlapping sequences of more than one Incyte Clone using a computer program for fragment assembly, such as the GELVIEW Fragment Assembly system (GCG, Madison, Wis.). Some sequences have been both extended and assembled to produce the consensus sequence.

As used herein, the term “correlates with expression of a polynucleotide” indicates that the detection of the presence of nucleic acids, the same or related to a nucleic acid sequence encoding MSP, by northern analysis is indicative of the presence of nucleic acids encoding MSP in a sample, and thereby correlates with expression of the transcript from the polynucleotide encoding MSP.

A “deletion,” as the term is used herein, refers to a change in the amino acid or nucleotide sequence that results in the absence of one or more amino acid residues or nucleotides.

The term “derivative,” as used herein, refers to the chemical modification of MSP, of a polynucleotide sequence encoding MSP, or of a polynucleotide sequence complementary to a polynucleotide sequence encoding MSP. Chemical modifications of a polynucleotide sequence can include, for example, replacement of hydrogen by an alkyl, acyl, or amino group. A derivative polynucleotide encodes a polypeptide which retains at least one biological or immunological function of the natural molecule. A derivative polypeptide is one modified by glycosylation, pegylation, or any similar process that retains at least one biological or immunological function of the polypeptide from which it was derived.

The term “homology,” as used herein, refers to a degree of complementarity. There may be partial homology or complete homology. The word “identity” may substitute for the word “homology.” A partially complementary sequence that at least partially inhibits an identical sequence from hybridizing to a target nucleic acid is referred to as “substantially homologous.” The inhibition of hybridization of the completely complementary sequence to the target sequence may be examined using a hybridization assay (Southern or northern blot, solution hybridization, and the like) under conditions of reduced stringency. A substantially homologous sequence or hybridization probe will compete for and inhibit the binding of a completely homologous sequence to the target sequence under conditions of reduced stringency. This is not to say that conditions of reduced stringency are such that non-specific binding is permitted, as reduced stringency conditions require that the binding of two sequences to one another be a specific (i.e., a selective) interaction. The absence of non-specific binding may be tested by the use of a second target sequence which lacks even a partial degree of complementarity (e.g., less than about 30% homology or identity). In the absence of non-specific binding, the substantially homologous sequence or probe will not hybridize to the second non-complementary target sequence.

The phrases “percent identity” or “% identity” refer to the percentage of sequence similarity found in a comparison of two or more amino acid or nucleic acid sequences. Percent identity can be determined electronically, e.g., by using the MegAlign program (DNASTAR, Inc., Madison Wis.). The MegAlign program can create alignments between two or more sequences according to different methods, e.g., the Clustal method. (See, e.g., Higgins, D. G. and P. M. Sharp (1988) Gene 73:237-244.) The Clustal algorithm groups sequences into clusters by examining the distances between all pairs. The clusters are aligned pairwise and then in groups. The percentage similarity between two amino acid sequences, e.g., sequence A and sequence B, is calculated by dividing the length of sequence A, minus the number of gap residues in sequence A, minus the number of gap residues in sequence B, into the sum of the residue matches between sequence A and sequence B, times one hundred. Gaps of low or of no homology between the two amino acid sequences are not included in determining percentage similarity. Percent identity between nucleic acid sequences can also be counted or calculated by other methods known in the art, e.g., the Jotun Hein method. (See, e.g., Hein, J. (1990) Methods Enzymol. 183:626-645.) Identity between sequences can also be determined by other methods known in the art, e.g., by varying hybridization conditions.

“Human artificial chromosomes” (HACs), as described herein, are linear microchromosomes which may contain DNA sequences of about 6 kb to 10 Mb in size, and which contain all of the elements required for stable mitotic chromosome segregation and maintenance. (See, e.g., Harrington, J. J. et al. (1997) Nat. Genet. 15:345-355.)

The term “humanized antibody,” as used herein, refers to antibody molecules in which the amino acid sequence in the non-antigen binding regions has been altered so that the antibody more closely resembles a human antibody, and still retains its original binding ability.

“Hybridization,” as the term is used herein, refers to any process by which a strand of nucleic acid binds with a complementary strand through base pairing.

As used herein, the term “hybridization complex” as used herein, refers to a complex formed between two nucleic acid sequences by virtue of the formation of hydrogen bonds between complementary bases. A hybridization complex may be formed in solution (e.g., C₀t or R₀t analysis) or formed between one nucleic acid sequence present in solution and another nucleic acid sequence immobilized on a solid support (e.g., paper, membranes, filters, chips, pins or glass slides, or any other appropriate substrate to which cells or their nucleic acids have been fixed).

The words “insertion” or “addition,” as used herein, refer to changes in an amino acid or nucleotide sequence resulting in the addition of one or more amino acid residues or nucleotides, respectively, to the sequence found in the naturally occurring molecule.

“Immune response” can refer to conditions associated with inflammation, trauma, immune disorders, or infectious or genetic disease, etc. These conditions can be characterized by expression of various factors, e.g., cytokines, chemokines, and other signaling molecules, which may affect cellular and systemic defense systems.

The term “microarray,” as used herein, refers to an arrangement of distinct polynucleotides arrayed on a substrate, e.g., paper, nylon or any other type of membrane, filter, chip, glass slide, or any other suitable solid support.

The terms “element” or “array element” as used herein in a microarray context, refer to hybridizable polynucleotides arranged on the surface of a substrate.

The term “modulate,” as it appears herein, refers to a change in the activity of MSP. For example, modulation may cause an increase or a decrease in protein activity, binding characteristics, or any other biological, functional, or immunological properties of MSP.

The phrases “nucleic acid” or “nucleic acid sequence,” as used herein, refer to an oligonucleotide, nucleotide, polynucleotide, or any fragment thereof, to DNA or RNA of genomic or synthetic origin which may be single-stranded or double-stranded and may represent the sense or the antisense strand, to peptide nucleic acid (PNA), or to any DNA-like or RNA-like material. In this context, “fragments” refers to those nucleic acid sequences which are greater than about 60 nucleotides in length, and most preferably are at least about 100 nucleotides, at least about 1000 nucleotides, or at least about 10,000 nucleotides in length.

The terms “operably associated” or “operably linked,” as used herein, refer to functionally related nucleic acid sequences. A promoter is operably associated or operably linked with a coding sequence if the promoter controls the transcription of the encoded polypeptide. While operably associated or operably linked nucleic acid sequences can be contiguous and in reading frame, certain genetic elements, e.g., repressor genes, are not contiguously linked to the encoded polypeptide but still bind to operator sequences that control expression of the polypeptide.

The term “oligonucleotide,” as used herein, refers to a nucleic acid sequence of at least about 6 nucleotides to 60 nucleotides, preferably about 15 to 30 nucleotides, and most preferably about 20 to 25 nucleotides, which can be used in PCR amplification or in a hybridization assay or microarray. As used herein, the term “oligonucleotide” is substantially equivalent to the terms “amplimer,” “primer,” “oligomer,” and “probe,” as these terms are commonly defined in the art.

“Peptide nucleic acid” (PNA), as used herein, refers to an antisense molecule or anti-gene agent which comprises an oligonucleotide of at least about 5 nucleotides in length linked to a peptide backbone of amino acid residues ending in lysine. The terminal lysine confers solubility to the composition. PNAs preferentially bind complementary single stranded DNA and RNA and stop transcript elongation, and may be pegylated to extend their lifespan in the cell. (See, e.g., Nielsen, P. E. et al. (1993) Anticancer Drug Des. 8:53-63.)

The term “sample,” as used herein, is used in its broadest sense. A biological sample suspected of containing nucleic acids encoding MSP, or fragments thereof, or MSP itself, may comprise a bodily fluid; an extract from a cell, chromosome, organelle, or membrane isolated from a cell; a cell; genomic DNA, RNA, or cDNA, in solution or bound to a solid support; a tissue; a tissue print; etc.

As used herein, the terms “specific binding” or “specifically binding” refer to that interaction between a protein or peptide and an agonist, an antibody, or an antagonist. The interaction is dependent upon the presence of a particular structure of the protein, e.g., the antigenic determinant or epitope, recognized by the binding molecule. For example, if an antibody is specific for epitope “A,” the presence of a polypeptide containing the epitope A, or the presence of free unlabeled A, in a reaction containing free labeled A and the antibody will reduce the amount of labeled A that binds to the antibody.

As used herein, the term “stringent conditions” refers to conditions which permit hybridization between polynucleotide sequences and the claimed polynucleotide sequences. Suitably stringent conditions can be defined by, for example, the concentrations of salt or formamide in the prehybridization and hybridization solutions, or by the hybridization temperature, and are well known in the art. In particular, stringency can be increased by reducing the concentration of salt, increasing the concentration of formamide, or raising the hybridization temperature.

For example, hybridization under high stringency conditions could occur in about 50% formamide at about 37° C. to 42° C. Hybridization could occur under reduced stringency conditions in about 35% to 25% formamide at about 30° C. to 35° C. In particular, hybridization could occur under high stringency conditions at 42° C. in 50% formamide, 5X SSPE, 0.3% SDS, and 200 μg/ml sheared and denatured salmon sperm DNA. Hybridization could occur under reduced stringency conditions as described above, but in 35% formamide at a reduced temperature of 35° C. The temperature range corresponding to a particular level of stringency can be further narrowed by calculating the purine to pyrimidine ratio of the nucleic acid of interest and adjusting the temperature accordingly. Variations on the above ranges and conditions are well known in the art.

The term “substantially purified,” as used herein, refers to nucleic acid or amino acid sequences that are removed from their natural environment and are isolated or separated, and are at least about 60% free, preferably about 75% free, and most preferably about 90% free from other components with which they are naturally associated.

A “substitution,” as used herein, refers to the replacement of one or more amino acids or nucleotides by different amino acids or nucleotides, respectively.

“Transformation,” as defined herein, describes a process by which exogenous DNA enters and changes a recipient cell. Transformation may occur under natural or artificial conditions according to various methods well known in the art, and may rely on any known method for the insertion of foreign nucleic acid sequences into a prokaryotic or eukaryotic host cell. The method for transformation is selected based on the type of host cell being transformed and may include, but is not limited to, viral infection, electroporation, heat shock, lipofection, and particle bombardment. The term “transformed” cells includes stably transformed cells in which the inserted DNA is capable of replication either as an autonomously replicating plasmid or as part of the host chromosome, as well as transiently transformed cells which express the inserted DNA or RNA for limited periods of time.

A “variant” of MSP, as used herein, refers to an amino acid sequence that is altered by one or more amino acids. The variant may have “conservative” changes, wherein a substituted amino acid has similar structural or chemical properties (e.g., replacement of leucine with isoleucine). More rarely, a variant may have “nonconservative” changes (e.g., replacement of glycine with tryptophan). Analogous minor variations may also include amino acid deletions or insertions, or both. Guidance in determining which amino acid residues may be substituted, inserted, or deleted without abolishing biological or immunological activity may be found using computer programs well known in the art, for example, DNASTAR software.

THE INVENTION

The invention is based on the discovery of new human membrane spanning proteins, collectively referred to as MSP and individually as MSP-1, MSP-2, MSP-3, MSP-4, MSP-5, MSP-6, MSP-7, MSP-8, MSP-9, MSP-10, MSP-11, MSP-12, MSP-13, MSP-14, MSP-15, MSP-16, and MSP-17; the polynucleotides encoding MSP (SEQ ID NO:18 through SEQ ID NO:34); and the use of these compositions for the diagnosis, treatment, or prevention of neoplastic, immunological, and reproductive disorders. Table 1 shows the sequence identification numbers, Incyte Clone identification number, cDNA library, public database (PD) homolog sequence identifier and homolog species description for each of the human membrane spanning proteins disclosed herein.

Nucleic acids encoding the MSP-1 of the present invention were first identified in Incyte Clone 77138 from the synovial membrane tissue cDNA library (SYNORAB01) using a computer search for amino acid sequence alignments. A consensus sequence, SEQ ID NO:18, was derived from Incyte Clones 77138 (SYNORAB01), 3576995 (BRONNOT01), 1995355 (BRSTTUT03), and 1260677 (SYNORAT05).

In one embodiment, the invention encompasses a polypeptide comprising the amino acid sequence of SEQ ID NO:1. MSP-1 is 238 amino acids in length and has two potential N glycosylation sites at residues N71 and N72; one potential cAMP- and cGMP-dependent protein kinase phosphorylation site at residue S233; three potential casein kinase II phosphorylation sites at residues T111, T131, and T195; four potential protein kinase C phosphorylation sites at residues T10, S44, T186, and S233; and the PMP-22/EMP/MP20 membrane protein family signature from about residue V205 to about residue M232; a proline-rich region from about P26 to about P41; and the G-protein alpha subunit signature from about R61 to about T76. MSP-1 shares 63% identity with the dog mucin-type gp40 protein (g1628360). In addition, the PMP-22/EMP/MP20 membrane protein family signature is conserved between these molecules. A fragment of SEQ ID NO:18 from about nucleotide 379 to about nucleotide 405 is useful for designing oligonucleotides or for use as a hybridization probe. Northern analysis shows the expression of this sequence in reproductive, nervous, and connective tissue cDNA libraries. Approximately 56% of these libraries are associated with neoplastic disorders and 23% with immune response.

Nucleic acids encoding the MSP-2 of the present invention were first identified in Incyte Clone 1381884 from the brain cDNA library (BRAITUT08) using a computer search for amino acid sequence alignments. A consensus sequence, SEQ ID NO:19, was derived

TABLE 1

| | | Clone | | | |
|---|---|---|---|---|---|
| Protein | Nucleotide | ID | Library | PD Homolog | Homolog species |
| SEQ ID NO:1 | SEQ ID NO:18 | 77138 | SYNORAIB01 | g1628360 | Canis familiaris |
| SEQ ID NO:2 | SEQ ID NO:19 | 1381884 | BRAITUT08 | g927071 | Homo sapiens |
| SEQ ID NO:3 | SEQ ID NO:20 | 1427590 | SINTBST01 | g2059326 | Mus musculus |
| SEQ ID NO:4 | SEQ ID NO:21 | 1457779 | COLNFET02 | g765256 | Homo sapiens |
| SEQ ID NO:5 | SEQ ID NO:22 | 1481261 | CORPNOT02 | g177900 | Homo sapiens |
| SEQ ID NO:6 | SEQ ID NO:23 | 1487802 | UCMCL5T01 | g2529739 | Homo sapiens |
| SEQ ID NO:7 | SEQ ID NO:24 | 1718830 | BLADNOT06 | g415907 | Saccharomyces cerevisiae |
| SEQ ID NO:8 | SEQ ID NO:25 | 1737775 | COLNNOT22 | g1184066 | Bos taurus |
| SEQ ID NO:9 | SEQ ID NO:26 | 1794154 | PROSTUT05 | WO9640907-A1 | Homo sapiens |
| SEQ ID NO:10 | SEQ ID NO:27 | 2027624 | KERANOT02 | g458939 | Saccharomyces cerevisiae |
| SEQ ID NO:11 | SEQ ID NO:28 | 2057213 | BEPINOT01 | g50599 | Mus musculus |
| SEQ ID NO:12 | SEQ ID NO:29 | 2073804 | ISLTNOT01 | g602370 | Saccharomyces cerevisiae |
| SEQ ID NO:13 | SEQ ID NO:30 | 2175401 | ENDCNOT03 | g624778 | Mus musculus |
| SEQ ID NO:14 | SEQ ID NO:31 | 2741580 | BRSTTUT14 | g2586350 | Homo sapiens |
| SEQ ID NO:15 | SEQ ID NO:32 | 2779610 | OVARTUT03 | g475006 | Homo sapiens |
| SEQ ID NO:16 | SEQ ID NO:33 | 2879792 | UTRSTUT05 | g309074 | Mus musculus |
| SEQ ID NO:17 | SEQ ID NO:34 | 3231062 | COTRNOT01 | g529228 | Felis catus |

from Incyte Clones 1381884 (BRAITUT08), 1862104 (PROSNOT19), 79810 (SYNORAB01), 2372040 (ADRENOT07), 1811401 (PROSTUT 12), 1811401 (PROSTUT12), and 1927265 (BRSTNOT02).

In one embodiment, the invention encompasses a polypeptide comprising the amino acid sequence of SEQ ID NO:2. MSP-2 is 83 amino acids in length and has one potential N glycosylation site at residue N80; one potential casein kinase II phosphorylation site at residue T71; and the inhibin beta A chain signature from about residue E39 to about residue K60. MSP-2 shares 51% identity with human thyroid receptor interactor (g927071). In addition, the C-terminal glycine residue spacings G41-{5N}-G47-{2N}-G50-{8N}-G59-{2N}-G62-{5N}-G69, where N is any amino acid, are conserved between these molecules. A fragment of SEQ ID NO:19 from about nucleotide 355 to about nucleotide 375 is useful for designing oligonucleotides or for use as a hybridization probe. Northern analysis shows the expression of this sequence in reproductive, cardiovascular, nervous, and gastrointestinal cDNA libraries. Approximately 43% of these libraries are associated with neoplastic disorders and 21% with immune response.

Nucleic acids encoding the MSP-3 of the present invention were first identified in Incyte Clone 1427590 from the ileum cDNA library (SINTBST01) using a computer search for amino acid sequence alignments. A consensus sequence, SEQ ID NO:20, was derived from Incyte Clones 1427590 (SINTBST01), 2535753 (BRAINOT18), 1728970 (BRSTTUT08), 1596989 (BRAINOT14), 1505713 (BRAITUT07), 918144 and 1967560 (BRSTNOT04), 2817264 (BRSTNOT14), 732336 (LUNGNOT03), and 519913 (MMLR2DT01).

In one embodiment, the invention encompasses a polypeptide comprising the amino acid sequence of SEQ ID NO:3. MSP-3 is 495 amino acids in length and has six potential N glycosylation sites at residues N28, N119, N161, N241, N295, and N413; seven potential casein kinase II phosphorylation sites at residues S110, T137, T216, S338, S368, S456, and T462; six potential protein kinase C phosphorylation sites at residues S322, S344, S392, S456, S467, and S499; and the dopamine 1B receptor signature from about residue Q54 to about residue W67. MSP-3 shares 98% identity with the mouse thymic epithelial cell surface antigen (g2059326). The cysteines at C145, C308, C496, C507, C510, and C515 are conserved across both molecules. A fragment of SEQ ID NO:20 from about nucleotide 1372 to about nucleotide 1392 is useful for designing oligonucleotides or for use as a hybridization probe. Northern analysis shows the expression of this sequence in reproductive, nervous, gastrointestinal, hematopoietic/immune, and cardiovascular cDNA libraries. Approximately 49% of these libraries are associated with neoplastic disorders and 21% with immune response.

Nucleic acids encoding the MSP-4 of the present invention were first identified in Incyte Clone 1457779 from the fetal colon cDNA library (COLNFET02) using a computer search for amino acid sequence alignments. A consensus sequence, SEQ ID NO:21, was derived from Incyte Clones 1457779 and 1457572 (COLNFET02), 2256441 (OVARTUT01), and the shotgun sequences SAGA01244, SAGA00103, SAGA01339, SAGA00583, and SAGA01282.

In one embodiment, the invention encompasses a polypeptide comprising the amino acid sequence of SEQ ID NO:4. MSP-4 is 495 amino acids in length and has seven potential N glycosylation sites at residues N67, N135, N304, N325, N363, N374, and N447; one potential cAMP- and cGMP-dependent protein kinase phosphorylation site at residue S136; four potential casein kinase II phosphorylation sites at residues S4, S153, T226, and T376; three potential protein kinase C phosphorylation sites at residues S4, S132, and T365; one potential tyrosine kinase phosphorylation site at residue Y392; a potential signal peptide sequence from about residue M1 to about G21; ten cysteine residues from about C78 to about C403; and four GDA1/CD39 family transmembrane signatures: TM1 extends from about F43 to about Y59; TM2, from about T118 to about L131; TM3, from about L162 to about L183; and TM4, from about G202 to about F215. MSP-4 shares 46% identity with human CD39 homolog (g765256). The cysteines at C78, C102, C246, C273, C292, C316, C329, C335, C381, and C403 are conserved across both molecules. In addition, the hydrophobic transmembrane domains are conserved between these molecules. A fragment of SEQ ID NO:21 from about nucleotide 1543 to about nucleotide 1566 is useful for designing oligonucleotides or for use as a hybridization probe. Northern analysis shows the expression of this sequence in gastrointestinal and ovarian cDNA libraries. Approximately 71% of these libraries are associated with neoplastic disorders and 14% with immune response.

Nucleic acids encoding the MSP-5 of the present invention were first identified in Incyte Clone 1481261 from the corpus callosum cDNA library (CORPNOT02) using a computer search for amino acid sequence alignments. A consensus sequence, SEQ ID NO:22, was derived from Incyte Clones 1481261 and 1476703 (CORPNOT02), and 2963217 (SCORNOT04).

In one embodiment, the invention encompasses a polypeptide comprising the amino acid sequence of SEQ ID NO:5. MSP-5 is 156 amino acids in length and has three potential casein kinase II phosphorylation sites at residues S3, T114, and S151; three potential protein kinase C phosphorylation sites at residues S3, S32, and T114; and a potential GPR1/FUN34/yaaH family transmembrane domain from about residue F95 to about residue I141. MSP-5 shares 25% identity with human differentiation-dependent intestinal A4 transmembrane protein (g177900). In addition, the hydrophobic transmembrane domain and one potential protein kinase C phosphorylation site are conserved between these molecules. A fragment of SEQ ID NO:22 from about nucleotide 373 to about nucleotide 396 is useful for designing oligonucleotides or for use as a hybridization probe. Northern analysis shows the expression of this sequence in brain, spinal cord, reproductive, muscle, and Alzheimer's disease brain tissue cDNA libraries. Approximately 64% of these libraries are associated with neoplastic disorders and 19% with immune response.

Nucleic acids encoding the MSP-6 of the present invention were first identified in Incyte Clone 1487802 from the umbilical cord mononuclear cell cDNA library (UCMCL5T01) using a computer search for amino acid sequence alignments. A consensus sequence, SEQ ID NO:23, was derived from Incyte Clones 1487802 (UCMCL5T01), 1802847 (COLNNOT27), 613194 (COLNTUT02), 875949 (LUNGAST01), 506242 (TMLR3DT01), and 1939036 (HIPONOT01).

In one embodiment, the invention encompasses a polypeptide comprising the amino acid sequence of SEQ ID NO:6. MSP-6 is 358 amino acids in length and has one potential N glycosylation site at residue N324; one potential cAMP- and cGMP-dependent protein kinase phosphorylation site at residue S340; six potential casein kinase II phosphorylation sites at residues S46, S143, S186, T260, T293, and S315; one potential glycosaminoglycan attachment site at residue S162; and six potential protein kinase C phosphorylation sites at residues T156, S170, S280, S339, S340, and S349. MSP-6 shares 95% identity with the human EBP50 protein (g2529739). A fragment of SEQ ID NO:23 from about nucleotide 487 to about nucleotide 516 is useful for designing oligonucleotides or for use as a hybridization probe. Northern analysis shows the expression of this sequence in reproductive, gastrointestinal, nervous, and hematopoietic/immunologic cDNA libraries. Approximately 51% of these libraries are associated with neoplastic disorders and 26% with immune response.

Nucleic acids encoding the MSP-7 of the present invention were first identified in Incyte Clone 1718830 from the bladder cDNA library (BLADNOT06) using a computer search for amino acid sequence alignments. A consensus sequence, SEQ ID NO:24, was derived from Incyte Clones 1718830 (BLADNOT06), 2057919 (BEPINOT01), 3419169 (UCMCNOT04), 3236861 (COLNUCT03), and 2585037 (BRAITUT22).

In one embodiment, the invention encompasses a polypeptide comprising the amino acid sequence of SEQ ID NO:7. MSP-7 is 280 amino acids in length and has one potential N glycosylation site at residue N250; four potential casein kinase II phosphorylation sites at residues T10, S70, S225, and S244; five potential protein kinase C phosphorylation sites at residues T10, S144, S236, S244, and T252; and the neutral zinc metallopeptidase zinc-binding region signature from about residue L80 to about residue V89. MSP-7 shares 21% identity with the Saccharomyces cerevisiae protein encoded by the YKR407 gene (g415907). In addition, the neutral zinc-binding region signature is conserved between these molecules. The fragment of SEQ ID NO:24 from about nucleotide 1006 to about nucleotide 1035 is useful for designing oligonucleotides or for use as a hybridization probe. Northern analysis shows the expression of this sequence in reproductive, urologic, cardiovascular, and hematopoietic/immunologic cDNA libraries. Approximately 41% of these libraries are associated with neoplastic disorders and 26% with immune response.

Nucleic acids encoding the MSP-8 of the present invention were first identified in Incyte Clone 1737775 from the colon cDNA library (COLNNOT22) using a computer search for amino acid sequence alignments. A consensus sequence, SEQ ID NO:25, was derived from Incyte Clones 1737775 and 1734248 (COLNNOT22), 1578253 (DUODNOT01), 2214423 (SINTFET03), 608819 (COLNNOT01), 1629002 and 1626949 (COLNPOT01), 1498226 and 1429362 (SINTBST01), 2925341 (SININOT04), and the shotgun sequences SAEC10115, SAEA00623, and SAEA00834.

In one embodiment, the invention encompasses a polypeptide comprising the amino acid sequence of SEQ ID NO:8. MSP-8 is 914 amino acids in length and has eight potential N glycosylation sites at residues N503, N585, N770, N804, N810, N831, N836, and N890; two potential cAMP- and cGMP-dependent protein kinase phosphorylation sites at residues T211 and T286; twelve potential casein kinase II phosphorylation sites at residues T87, S245, S271, S364, S366, T411, T597, T652, T663, S795, T870, and S876; one potential glycosaminoglycan attachment site at residue S477; thirteen potential protein kinase C phosphorylation sites at residues T68, T84, T98, T207, T232, S366, S483, T563, T580, T594, T597, T601, and S672; eleven cysteine residues from about C125 to about C421; a potential signal peptide sequence from about residue M1 to about S21; and a cadherin family signature from about N29 to about P44. MSP-8 shares 53% identity with the bovine calcium-activated chloride channel (g 1184066). The cysteines at C125, C187, C200, C205, C210, C223, C250, C267, C308, C386, and C421 are conserved across both molecules. A fragment of SEQ ID NO:25 from about nucleotide 1447 to about nucleotide 1485 is useful for designing oligonucleotides or for use as a hybridization probe. Northern analysis shows the expression of this sequence in gastrointestinal, female reproductive, and cardiovascular cDNA libraries. Approximately 32% of these libraries are associated with neoplastic disorders and 56% with immune response.

Nucleic acids encoding the MSP-9 of the present invention were first identified in Incyte Clone 1794154 from the prostate cDNA library (PROSTUT05) using a computer search for amino acid sequence alignments. A consensus sequence, SEQ ID NO:26, was derived from Incyte Clones 1794154 (PROSTUT05), 3116657 (LUNGTUT13), 1726333 (PROSNOT14), 3217158 (TESTNOT07), 1435295 (PANCNOT08), 2530829 (GBLANOT02), 728143 (SYNOOAT01), 1539910 (SINTTUT01), 1467860 (PANCTUT02), 1484333 (CORPNOT02), 1927469 (BRSTNOT02), 1516116 (PANCTUT01), 1571055 (UTRSNOT05), 1965064 (BRSTNOT04), and 2683922 (LUNGNOT23).

In one embodiment, the invention encompasses a polypeptide comprising the amino acid sequence of SEQ ID NO:9. MSP-9 is 950 amino acids in length and has three potential N glycosylation site at residues N667 N668 N835; six potential cAMP- and cGMP-dependent protein kinase phosphorylation sites at residues S79, T204, S409, S434, S658, and S767; seventeen potential casein kinase II phosphorylation sites at residues S64, T65, S113, S284, T398, S409, S418, T437, T449, S518, S576, T591, S611, T708, T717, S735, and S838; twenty five potential protein kinase C phosphorylation sites at residues T2, T28, S45, T55, S113, S124, S308, T347, T365, T384, S393, T404, S418, T429, T430, S454, S537, S565, S572, S583, T600, S607, T654, T670, and T732; one potential tyrosine kinase phosphorylation site at residue Y918; and a potential signal peptide sequence from about M1 to P23. MSP-9 shares 28% identity with human type I, p80 IL-1-receptor intracellular domain ligand (WO9640907-A1). A fragment of SEQ ID NO:26 from about nucleotide 3220 to about nucleotide 3243 is useful for designing oligonucleotides or for use as a hybridization probe. Northern analysis shows the expression of this sequence in gastrointestinal, male reproductive, and muscle cDNA libraries. Approximately 54% of these libraries are associated with neoplastic disorders and 22% with immune response.

Nucleic acids encoding the MSP-10 of the present invention were first identified in Incyte Clone 2027624 from the breast epidermal keratinocyte cDNA library (KERANOT02) using a computer search for amino acid sequence alignments. A consensus sequence, SEQ ID NO:27, was derived from Incyte Clones 2027624 (KERANOT02), 3616164 (EPIPNOT01), 2492955 (ADRETUT05), 1557177 (BLADTUT04), 1466958 (PANCTUT02), 842091 (PROSTUT05), 1305186 (PLACNOT02), 1871863 (LEUKNOT02), 1345853 (PROSNOT11), and 1351147 (LATRTUT02).

In one embodiment, the invention encompasses a polypeptide comprising the amino acid sequence of SEQ ID NO:10. MSP-10 is 578 amino acids in length and has three potential N glycosylation sites at residues N164, N291, and N327; nine potential casein kinase II phosphorylation sites at residues S29, T52, S81, S108, T119, T121, S126, T267, and T303; one potential glycosaminoglycan attachment site at residue S359; seven potential protein kinase C phosphorylation sites at residues S29, S81, S126, S156, S196, T311, and T403; one potential tyrosine kinase phosphorylation site at residue Y287; and one potential signal peptide sequence from about M1 to about A23. MSP-10 shares 24% identity with Saccharomyces cerevisiae protein encoded by YHR188c gene (g458939). Fragments of SEQ ID NO:27 from about nucleotide 1078 to about nucleotide 1101 and from about nucleotide 1585 to about nucleotide 1614 are useful for designing oligonucleotides or for use as a hybridization probe. Northern analysis shows the expression of this sequence in reproductive, gastrointestinal, nervous, and cardiovascular cDNA libraries. Approximately 50% of these libraries are associated with neoplastic disorders and 24% with immune response.

Nucleic acids encoding the MSP-11 of the present invention were first identified in Incyte Clone 2057213 from the bronchial epithelium cell line cDNA library (BEPINOT01) using a computer search for amino acid sequence alignments. A consensus sequence, SEQ ID NO:28, was derived from Incyte Clones 2057213 (BEPINOT01), 2252506 (OVARTUT01), 3431316 (SKINNOT04), and 2670856 (ESOGTUT02).

In one embodiment, the invention encompasses a polypeptide comprising the amino acid sequence of SEQ ID NO:11. MSP-11 is 270 amino acids in length and has one potential casein kinase II phosphorylation site at residue T202; two potential protein kinase C phosphorylation sites at residues T182 and T238; four potential membrane spanning domains: TM1 extends from about Q48 to about I71; TM2, from about I74 to about Y97; TM3, from about G125 to about 154; and TM4, from about A165 to about L209; and the connexin 1 signature from about C53 to about D66 and the connexin 2 signature from about C164 to about P181. MSP-11 shares 81% identity with mouse connexin 31 (g50599). In addition, the hydrophobic transmembrane domains are conserved between these molecules. A fragment of SEQ ID NO:28 from about nucleotide 559 to about nucleotide 588 is useful for designing oligonucleotides or for use as a hybridization probe. Northern analysis shows the expression of this sequence in gastrointestinal, reproductive, and bronchial epithelial cell line cDNA libraries. Approximately 40% of these libraries are associated with neoplastic disorders.

Nucleic acids encoding the MSP-12 of the present invention were first identified in Incyte Clone 2073804 from the pancreatic islelet cDNA library (ISLTNOT01) using a computer search for amino acid sequence alignments. A consensus sequence, SEQ ID NO:29, was derived from Incyte Clones 2073804 (ISLTNOT01) and 1982628 (LUNGTUT03).

In one embodiment, the invention encompasses a polypeptide comprising the amino acid sequence of SEQ ID NO:12. MSP-12 is 154 amino acids in length and has two potential casein kinase II phosphorylation sites at residues T60 and T82; two potential glycosaminoglycan attachment sites at residues S12 and S14; four potential protein kinase C phosphorylation sites at residues S5, T60, T67, and T82; and four potential transmembrane regions: TM1 from about K18 to about F30; TM2 from about H54 to about L61; TM3 from about M73 to about V83; and TM4 from about K145 to about S154. MSP-12 shares 32% identity with the Saccharomyces cerevisiae potential transmembrane protein encoded by the Ye10003wp gene (g602370). A fragment of SEQ ID NO:29 from about nucleotide 223 to about nucleotide 252 is useful for designing oligonucleotides or for use as a hybridization probe. Northern analysis shows the expression of this sequence in cardiovascular, reproductive, and nervous tissue cDNA libraries. Approximately 62% of these libraries are associated with neoplastic disorders and 31% with immune response.

Nucleic acids encoding the MSP-13 of the present invention were first identified in Incyte Clone 2175401 from the dermal microvascular endothelial cell cDNA library (ENDCNOT03) using a computer search for amino acid sequence alignments. A consensus sequence, SEQ ID NO:30, was derived from Incyte Clones 2175401 (ENDCNOT03), 428432 (BLADNOT01), 2175401 (ENDCNOT03), 79481 (SYNORAB01), and 415866 (BRSTNOT01).

In one embodiment, the invention encompasses a polypeptide comprising the amino acid sequence of SEQ ID NO:13. MSP-13 is 263 amino acids in length and has one potential N glycosylation site at residue N166; three potential casein kinase II phosphorylation sites at residues T35, S132, and S134; and six potential protein kinase C phosphorylation sites at residues T28, T35, S50, S100, S185, and S224. MSP-13 shares 95% identity with the mouse E25 integral membrane protein (g624778). A fragment of SEQ ID NO:30 from about nucleotide 415 to about nucleotide 444 is useful for designing oligonucleotides or for use as a hybridization probe. Northern analysis shows the expression of this sequence in reproductive, nervous, cardiovascular, muscle, and in Alzheimer's disease brain cDNA libraries. Approximately 41% of these libraries are associated with neoplastic disorders and 30% with immune response.

Nucleic acids encoding the MSP-14 of the present invention were first identified in Incyte Clone 2741580 from the breast cDNA library (BRSTTUT14) using a computer search for amino acid sequence alignments. A consensus sequence, SEQ ID NO:31, was derived from Incyte Clones 2741580 (BRSTTUT14), 2672813, (KIDNNOT19), 779308 (MYOMNOT01), and 1996070 (BRSTTUT03).

In one embodiment, the invention encompasses a polypeptide comprising the amino acid sequence of SEQ ID NO:14. MSP-14 is 240 amino acids in length and has one potential N glycosylation site at residue N180; one potential casein kinase II phosphorylation site at residue T164; one potential protein kinase C phosphorylation site at residue T233; and the TM4SF protein family signature from about residue G61 to about residue L83. In addition, there are four potential membrane spanning domains: TM1 extends from about L10 to about L34; TM2, from about S50 to about I76; TM3, from about K77 to about F105; and TM4, from about V204 to about I230. MSP-14 shares 54% identity with human NAG-2 TM4SF protein (g2586350). In addition, the transmembrane domains are conserved between these molecules. A fragment of SEQ ID NO:31 from about nucleotide 316 to about nucleotide 348 is useful for designing oligonucleotides or for use as a hybridization probe. Northern analysis shows the expression of this sequence in gastrointestinal, reproductive, and urologic cDNA libraries. Approximately 58% of these libraries are associated with neoplastic disorders and 33% with immune response.

Nucleic acids encoding the MSP-15 of the present invention were first identified in Incyte Clone 2779610 from the ovarian cDNA library (OVARTUT03) using a computer search for amino acid sequence alignments. A consensus sequence, SEQ ID NO:32, was derived from Incyte Clones 2779610 (OVARTUT03), 260635 and 483040 (HNT2RAT01), 762225 (BRAITUT02), 2014257 (TESTNOT03), 833639 (PROSNOT07), and 414915 (BRSTNOT01).

In one embodiment, the invention encompasses a polypeptide comprising the amino acid sequence of SEQ ID NO:15. MSP-15 is 245 amino acids in length and has one potential N glycosylation site at residue N134; two potential casein kinase II phosphorylation sites at residues S135 and T203; four potential protein kinase C phosphorylation sites at residues S5, T82, S121, and S169; one potential tyrosine kinase phosphorylation site at residue Y167; and the TM4SF protein family signature from about residue G66 to about residue I237. In addition, there are three potential membrane spanning domians: TM1 extends from about T15 to about G42; TM2, from about V63 to about F112; TM3, from about I204 to about T238. MSP-15 shares 48% identity with human TALLA-1 TM4SF protein (g475006). In addition, the transmembrane domains are conserved between these molecules. A fragment of SEQ ID NO:32 from about nucleotide 319 to about nucleotide 351 is useful for designing oligonucleotides or for use as a hybridization probe. Northern analysis shows the expression of this sequence in gastrointestinal, reproductive, nervous, and urologic cDNA libraries. Approximately 55% of these libraries are associated with neoplastic disorders and 16% with immune response.

Nucleic acids encoding the MSP-16 of the present invention were first identified in Incyte Clone 2879792 from the uterus cDNA library (UTRSTUT05) using a computer search for amino acid sequence alignments. A consensus sequence, SEQ ID NO:33, was derived from Incyte Clones 2879792 (UTRSTUT05), 693310 (LUNGNOT23), 358525 (SYNORAB01), and 1822710 (GBLATUT01).

In one embodiment, the invention encompasses a polypeptide comprising the amino acid sequence of SEQ ID NO:16. MSP-16 is 275 amino acids in length and has one potential N glycosylation site at residue N140; one potential casein kinase II phosphorylation site at residue S271; a potential signal peptide sequence from about residue M1 to about residue S25; and potential transmembrane regions from about residue L-79 to about residue C112 and from about residue L148 to about residue A167. MSP-16 shares 64% identity with the mouse 19.5 cell surface protein (g309074). In addition, the hydrophobic transmembrane domains are conserved between these molecules. A fragment of SEQ ID NO:33 from about nucleotide 607 to about nucleotide 639 is useful for designing oligonucleotides or for use as a hybridization probe. Northern analysis shows the expression of this sequence in gastrointestinal, reproductive, nervous, dermatological, and cardiovascular cDNA libraries. Approximately 43% of these libraries are associated with neoplastic disorders and 23% with immune response.

Nucleic acids encoding the MSP-17 of the present invention were first identified in Incyte Clone 3231062 from the transverse colon cDNA library (COTRNOT01) using a computer search for amino acid sequence alignments. A consensus sequence, SEQ ID NO:34, was derived from Incyte Clones 3231062 (COTRNOT01), 3403088 (ESOGNOT03), 3523907 (ESOGTUN01), 3706862 and 3704223 (PENCNOT07), 3201831 (PENCNOT02), and 1555684 (BLADTUT04).

In one embodiment, the invention encompasses a polypeptide comprising the amino acid sequence of SEQ ID NO:17. MSP-17 is 222 amino acids in length and has one potential N glycosylation site at residue N139; two potential casein kinase II phosphorylation sites at residues S46 and T122; one potential protein kinase C phosphorylation site at residue S183; one potential tyrosine kinase phosphorylation site at residue Y56; and the TM4SF protein family signature from about residue G66 to about residue L88. In addition, there are four potential transmembrane domains: TM1 extends from about Y13 to about F36; TM2, from about F55 to about M76; TM3, from about R82 to about F110; and TM4, from about L188 to about I203. MSP-17 shares 46% identity with the feline CD9 TM4SF protein (g529228). In addition, the hydrophobic transmembrane domains are conserved between these molecules. A fragment of SEQ ID NO:34 from about nucleotide 595 to about nucleotide 621 is useful for designing oligonucleotides or for use as a hybridization probe. Northern analysis shows the expression of this sequence in gastrointestinal, male reproductive, and muscle cDNA libraries. Approximately 55% of these libraries are associated with neoplastic disorders and 18% with immune response.

The invention also encompasses MSP variants. A preferred MSP variant is one which has at least about 80%, more preferably at least about 90%, and most preferably at least about 95% amino acid sequence identity to the MSP amino acid sequence, and which contains at least one functional or structural characteristic of MSP.

The invention also encompasses polynucleotides which encode MSP. Accordingly, any nucleic acid sequence which encodes the amino acid sequence of MSP can be used to produce recombinant molecules which express MSP. In a particular embodiment, the invention encompasses a polynucleotide consisting of a nucleic acid sequence selected from the group consisting of SEQ ID NO:18 through SEQ ID NO:34.

The invention also encompasses a variant of a polynucleotide sequence encoding MSP. In particular, such a variant polynucleotide sequence will have at least about 80%, more preferably at least about 90%, and most preferably at least about 95% polynucleotide sequence identity to the polynucleotide sequence encoding MSP. A particular aspect of the invention encompasses a variant of a nucleic acid sequence selected from the group consisting of SEQ ID NO:18 through SEQ ID NO:34 which has at least about 80%, more preferably at least about 90%, and most preferably at least about 95% polynucleotide consisting of a nucleic acid sequence selected from the group consisting of SEQ ID NO:18 through SEQ ID NO:34. Any one of the polynucleotide variants described above can encode an amino acid sequence which contains at least one functional or structural characteristic of MSP.

It will be appreciated by those skilled in the art that as a result of the degeneracy of the genetic code, a multitude of polynucleotide sequences encoding MSP, some bearing minimal homology to the polynucleotide sequences of any known and naturally occurring gene, may be produced. Thus, the invention contemplates each and every possible variation of polynucleotide sequence that could be made by selecting combinations based on possible codon choices. These combinations are made in accordance with the standard triplet genetic code as applied to the polynucleotide sequence of naturally occurring MSP, and all such variations are to be considered as being specifically disclosed.

Although nucleotide sequences which encode MSP and its variants are preferably capable of hybridizing to the nucleotide sequence of the naturally occurring MSP under appropriately selected conditions of stringency, it may be advantageous to produce nucleotide sequences encoding MSP or its derivatives possessing a substantially different codon usage. Codons may be selected to increase the rate at which expression of the peptide occurs in a particular prokaryotic or eukaryotic host in accordance with the frequency with which particular codons are utilized by the host. Other reasons for substantially altering the nucleotide sequence encoding MSP and its derivatives without altering the encoded amino acid sequences include the production of RNA transcripts having more desirable properties, such as a greater half-life, than transcripts produced from the naturally occurring sequence.

The invention also encompasses production of DNA sequences which encode MSP and MSP derivatives, or fragments thereof, entirely by synthetic chemistry. After production, the synthetic sequence may be inserted into any of the many available expression vectors and cell systems using reagents that are well known in the art. Moreover, synthetic chemistry may be used to introduce mutations into a sequence encoding MSP or any fragment thereof.

Also encompassed by the invention are polynucleotide sequences that are capable of hybridizing to the claimed nucleotide sequences, and in particular, those shown in SEQ ID NO:18 through SEQ ID NO:34, and fragments thereof, under various conditions of stringency. (See, e.g., Wahl, G. M. and S. L. Berger (1987) Methods Enzymol. 152:399-407; and Kimmel, A. R. (1987) Methods Enzymol. 152:507-511.)

Methods for DNA sequencing are well known and generally available in the art and may be used to practice any of the embodiments of the invention. The methods may employ such enzymes as the Klenow fragment of DNA polymerase I, Sequenase® (US Biochemical Corp., Cleveland, Ohio), Taq polymerase (Perkin Elmer), thermostable T7 polymerase (Amersham, Chicago, Ill.), or combinations of polymerases and proofreading exonucleases such as those found in the ELONGASE Amplification System (GIBCO/BRL, Gaithersburg, Md.). Preferably, the process is automated with machines such as the Hamilton Micro Lab 2200 (Hamilton, Reno, Nev.), Peltier Thermal Cycler (PTC200; MJ Research, Watertown, Mass.) and the ABI Catalyst and 373 and 377 DNA Sequencers (Perkin Elmer).

The nucleic acid sequences encoding MSP may be extended utilizing a partial nucleotide sequence and employing various methods known in the art to detect upstream sequences, such as promoters and regulatory elements. For example, one method which may be employed, restriction-site PCR, uses universal primers to retrieve unknown sequence adjacent to a known locus. (See, e.g., Sarkar, G. (1993) PCR Methods Applic. 2:318-322.) In particular, genomic DNA is first amplified in the presence of a primer which is complementary to a linker sequence within the vector and a primer specific to a region of the nucleotide sequenc. The amplified sequences are then subjected to a second round of PCR with the same linker primer and another specific primer internal to the first one. Products of each round of PCR are transcribed with an appropriate RNA polymerase and sequenced using reverse transcriptase.

Inverse PCR may also be used to amplify or extend sequences using divergent primers based on a known region. (See, e.g., Triglia, T. et al. (1988) Nucleic Acids Res. 16:8186.) The primers may be designed using commercially available software such as OLIGO 4.06 Primer Analysis software (National Biosciences Inc., Plymouth, Minn.) or another appropriate program to be about 22 to 30 nucleotides in length, to have a GC content of about 50% or more, and to anneal to the target sequence at temperatures of about 68° C. to 72° C. The method uses several restriction enzymes to generate a suitable fragment in the known region of a gene. The fragment is then circularized by intramolecular ligation and used as a PCR template.

Another method which may be used is capture PCR, which involves PCR amplification of DNA fragments adjacent to a known sequence in human and yeast artificial chromosome DNA. (See, e.g., Lagerstrom, M. et al. (1991) PCR Methods Applic. 1:111-119.) In this method, multiple restriction enzyme digestions and ligations may be used to place an engineered double-stranded sequence into an unknown fragment of the DNA molecule before performing PCR. Other methods which may be used to retrieve unknown sequences are known in the art. (See, e.g., Parker, J. D. et al. (1991) Nucleic Acids Res. 19:3055-3060.) Additionally, one may use PCR, nested primers, and PROMOTERFINDER libraries to walk genomic DNA (Clontech, Palo Alto, Calif.). This process avoids the need to screen libraries and is useful in finding intron/exon junctions.

When screening for full-length cDNAs, it is preferable to use libraries that have been size-selected to include larger cDNAs. Also, random-primed libraries are preferable in that they will include more sequences which contain the 5′ regions of genes. Use of a randomly primed library may be especially preferable for situations in which an oligo d(T) library does not yield a full-length cDNA. Genomic libraries may be useful for extension of sequence into 5′ non-transcribed regulatory regions.

Capillary electrophoresis systems which are commercially available may be used to analyze the size or confirm the nucleotide sequence of sequencing or PCR products. In particular, capillary sequencing may employ flowable polymers for electrophoretic separation, four different fluorescent dyes (one for each nucleotide) which are laser activated, and a charge coupled device camera for detection of the emitted wavelengths. Output/light intensity may be converted to electrical signal using appropriate software (e.g., GENOTYPER and SEQUENCE NAVIGATOR, Perkin Elmer), and the entire process from loading of samples to computer analysis and electronic data display may be computer controlled. Capillary electrophoresis is especially preferable for the sequencing of small pieces of DNA which might be present in limited amounts in a particular sample.

In another embodiment of the invention, polynucleotide sequences or fragments thereof which encode MSP may be used in recombinant DNA molecules to direct expression of MSP, or fragments or functional equivalents thereof, in appropriate host cells. Due to the inherent degeneracy of the genetic code, other DNA sequences which encode substantially the same or a functionally equivalent amino acid sequence may be produced, and these sequences may be used to clone and express MSP.

As will be understood by those of skill in the art, it may be advantageous to produce MSP-encoding nucleotide sequences possessing non-naturally occurring codons. For example, codons preferred by a particular prokaryotic or eukaryotic host can be selected to increase the rate of protein expression or to produce an RNA transcript having desirable properties, such as a half-life which is longer than that of a transcript generated from the naturally occurring sequence.

The nucleotide sequences of the present invention can be engineered using methods generally known in the art in order to alter MSP-encoding sequences for a variety of reasons including, but not limited to, alterations which modify the cloning, processing, and/or expression of the gene product. DNA shuffling by random fragmentation and PCR reassembly of gene fragments and synthetic oligonucleotides may be used to engineer the nucleotide sequences. For example, site-directed mutagenesis may be used to insert new restriction sites, alter glycosylation patterns, change codon preference, produce splice variants, introduce mutations, and so forth.

In another embodiment of the invention, natural, modified, or recombinant nucleic acid sequences encoding MSP may be ligated to a heterologous sequence to encode a fusion protein. For example, to screen peptide libraries for inhibitors of MSP activity, it may be useful to encode a chimeric MSP protein that can be recognized by a commercially available antibody. A fusion protein may also be engineered to contain a cleavage site located between the MSP encoding sequence and the heterologous protein sequence, so that MSP may be cleaved and purified away from the heterologous moiety.

In another embodiment, sequences encoding MSP may be synthesized, in whole or in part, using chemical methods well known in the art. (See, e.g., Caruthers, M. H. et al. (1980) Nucleic Acids Symp. Ser. 7:215-223, and Horn, T. et al. (1980) Nucleic Acids Symp. Ser. 7:225-232.) Alternatively, the protein itself may be produced using chemical methods to synthesize the amino acid sequence of MSP, or a fragment thereof. For example, peptide synthesis can be performed using various solid-phase techniques. (See, e.g., Roberge, J. Y. et al. (1995) Science 269:202-204.) Automated synthesis may be achieved using the ABI 431A Peptide Synthesizer (Perkin Elmer). Additionally, the amino acid sequence of MSP, or any part thereof, may be altered during direct synthesis and/or combined with sequences from other proteins, or any part thereof, to produce a variant polypeptide.

The peptide may be substantially purified by preparative high performance liquid chromatography. (See, e.g, Chiez, R. M. and F. Z. Regnier (1990) Methods Enzymol. 182:392-421.) The composition of the synthetic peptides may be confirmed by amino acid analysis or by sequencing. (See, e.g., Creighton, T. (1983) Proteins, Structures and Molecular Properties, WH Freeman and Co., New York N.Y.)

In order to express a biologically active MSP, the nucleotide sequences encoding MSP or derivatives thereof may be inserted into appropriate expression vector, i.e., a vector which contains the necessary elements for the transcription and translation of the inserted coding sequence.

Methods which are well known to those skilled in the art may be used to construct expression vectors containing sequences encoding MSP and appropriate transcriptional and translational control elements. These methods include in vitro recombinant DNA techniques, synthetic techniques, and in vivo genetic recombination. (See, e.g., Sambrook, J. et al. (1989) Molecular Cloning, A Laboratory Manual, Cold Spring Harbor Press, Plainview N.Y., ch. 4, 8, and 16-17; and Ausubel, F. M. et al. (1995, and periodic supplements) Current Protocols in Molecular Biology, John Wiley & Sons, New York N.Y., ch. 9, 13, and 16.)

A variety of expression vector/host systems may be utilized to contain and express sequences encoding MSP. These include, but are not limited to, microorganisms such as bacteria transformed with recombinant bacteriophage, plasmid, or cosmid DNA expression vectors; yeast transformed with yeast expression vectors; insect cell systems infected with virus expression vectors (e.g., baculovirus); plant cell systems transformed with virus expression vectors (e.g., cauliflower mosaic virus (CaMV) or tobacco mosaic virus (TMV)) or with bacterial expression vectors (e.g., Ti or pBR322 plasmids); or animal cell systems. The invention is not limited by the host cell employed.

The “control elements” or “regulatory sequences” are those non-translated regions, e.g., enhancers, promoters, and 5′ and 3′ untranslated regions, of the vector and polynucleotide sequences encoding MSP which interact with host cellular proteins to carry out transcription and translation. Such elements may vary in their strength and specificity. Depending on the vector system and host utilized, any number of suitable transcription and translation elements, including constitutive and inducible promoters, may be used. For example, when cloning in bacterial systems, inducible promoters, e.g., hybrid lacZ promoter of the BLUESCRIPT phagemid (Stratagene, La Jolla, Calif.) or PSPORT1 plasmid (GIBCO/BRL), may be used. The baculovirus polyhedrin promoter may be used in insect cells. Promoters or enhancers derived from the genomes of plant cells (e.g., heat shock, RUBISCO, and storage protein genes) or from plant viruses (e.g., viral promoters or leader sequences) may be cloned into the vector. In mammalian cell systems, promoters from mammalian genes or from mammalian viruses are preferable. If it is necessary to generate a cell line that contains multiple copies of the sequence encoding MSP, vectors based on SV40 or EBV may be used with an appropriate selectable marker.

In bacterial systems, a number of expression vectors may be selected depending upon the use intended for MSP. For example, when large quantities of MSP are needed for the induction of antibodies, vectors which direct high level expression of fusion proteins that are readily purified may be used. Such vectors include, but are not limited to, multifunctional E. coli cloning and expression vectors such as BLUESCRIPT (Stratagene), in which the sequence encoding MSP may be ligated into the vector in frame with sequences for the amino-terminal Met and the subsequent 7 residues of β-galactosidase so that a hybrid protein is produced, and pIN vectors. (See, e.g., Van Heeke, G. and S. M. Schuster (1989) J. Biol. Chem. 264:5503-5509.) pGEX vectors (Pharmacia Biotech, Uppsala, Sweden) may also be used to express foreign polypeptides as fusion proteins with glutathione S-transferase (GST). In general, such fusion proteins are soluble and can easily be purified from lysed cells by adsorption to glutathione-agarose beads followed by elution in the presence of free glutathione. Proteins made in such systems may be designed to include heparin, thrombin, or factor XA protease cleavage sites so that the cloned polypeptide of interest can be released from the GST moiety at will.

In the yeast Saccharomyces cerevisiae, a number of vectors containing constitutive or inducible promoters, such as alpha factor, alcohol oxidase, and PGH, may be used. (See, e.g., Ausubel, supra; and Bitter, G. A. et al. (1987) Methods Enzymol. 153:516-544.)

In cases where plant expression vectors are used, the expression of sequences encoding MSP may be driven by any of a number of promoters. For example, viral promoters such as the 35S and 19S promoters of CaMV may be used alone or in combination with the omega leader sequence from TMV. (Takamatsu, N. (1987) EMBO J. 6:307-311.) Alternatively, plant promoters such as the small subunit of RUBISCO or heat shock promoters may be used. (See, e.g., Coruzzi, G. et al. (1984) EMBO J. 3:1671-1680; Broglie, R. et al. (1984) Science 224:838-843; and Winter, J. et al. (1991) Results Probl. Cell Differ. 17:85-105.) These constructs can be introduced into plant cells by direct DNA transformation or pathogen-mediated transfection. Such techniques are described in a number of generally available reviews. (See, e.g., Hobbs, S. or Murry, L. E. in McGraw Hill Yearbook of Science and Technology (1992) McGraw Hill, New York N.Y.; pp. 191-196.)

An insect system may also be used to express MSP. For example, in one such system, Autographa californica nuclear polyhedrosis virus (AcNPV) is used as a vector to express foreign genes in Spodoptera frugiperda cells or in Trichoplusia larvae. The sequences encoding MSP may be cloned into a non-essential region of the virus, such as the polyhedrin gene, and placed under control of the polyhedrin promoter. Successful insertion of sequences encoding MSP will render the polyhedrin gene inactive and produce recombinant virus lacking coat protein. The recombinant viruses may then be used to infect, for example, S. frugiperda cells or Trichoplusia larvae in which MSP may be expressed. (See, e.g., Engelhard, E. K. et al. (1994) Proc. Natl. Acad. Sci. USA 91:3224-3227.)

In mammalian host cells, a number of viral-based expression systems may be utilized. In cases where an adenovirus is used as an expression vector, sequences encoding MSP may be ligated into an adenovirus transcription/translation complex consisting of the late promoter and tripartite leader sequence. Insertion in a non-essential E1 or E3 region of the viral genome may be used to obtain a viable virus which is capable of expressing MSP in infected host cells. (See, e.g., Logan, J. and T. Shenk (1984) Proc. Natl. Acad. Sci. USA 81:3655-3659.) In addition, transcription enhancers, such as the Rous sarcoma virus (RSV) enhancer, may be used to increase expression in mammalian host cells.

Human artificial chromosomes (HACs) may also be employed to deliver larger fragments of DNA than can be contained and expressed in a plasmid. HACs of about 6 kb to 10 Mb are constructed and delivered via conventional delivery methods (liposomes, polycationic amino polymers, or vesicles) for therapeutic purposes.

Specific initiation signals may also be used to achieve more efficient translation of sequences encoding MSP. Such signals include the ATG initiation codon and adjacent sequences. In cases where sequences encoding MSP and its initiation codon and upstream sequences are inserted into the appropriate expression vector, no additional transcriptional or translational control signals may be needed. However, in cases where only coding sequence, or a fragment thereof, is inserted, exogenous translational control signals including the ATG initiation codon should be provided. Furthermore, the initiation codon should be in the correct reading frame to ensure translation of the entire insert. Exogenous translational elements and initiation codons may be of various origins, both natural and synthetic. The efficiency of expression may be enhanced by the inclusion of enhancers appropriate for the particular cell system used. (See, e.g., Scharf, D. et al. (1994) Results Probl. Cell Differ. 20:125-162.)

In addition, a host cell strain may be chosen for its ability to modulate expression of the inserted sequences or to process the expressed protein in the desired fashion. Such modifications of the polypeptide include, but are not limited to, acetylation, carboxylation, glycosylation, phosphorylation, lipidation, and acylation. Post-translational processing which cleaves a “prepro” form of the protein may also be used to facilitate correct insertion, folding, and/or function. Different host cells which have specific cellular machinery and characteristic mechanisms for post-translational activities (e.g., CHO, HeLa, MDCK, HEK293, and WI38), are available from the American Type Culture Collection (ATCC, Manassas Va.) and may be chosen to ensure the correct modification and processing of the foreign protein.

For long term, high yield production of recombinant proteins, stable expression is preferred. For example, cell lines capable of stably expressing MSP can be transformed using expression vectors which may contain viral origins of replication and/or endogenous expression elements and a selectable marker gene on the same or on a separate vector. Following the introduction of the vector, cells may be allowed to grow for about 1 to 2 days in enriched media before being switched to selective media. The purpose of the selectable marker is to confer resistance to selection, and its presence allows growth and recovery of cells which successfully express the introduced sequences. Resistant clones of stably transformed cells may be proliferated using tissue culture techniques appropriate to the cell type.

Any number of selection systems may be used to recover transformed cell lines. These include, but are not limited to, the herpes simplex virus thymidine kinase genes and adenine phosphoribosyltransferase genes, which can be employed in tk and apr cells, respectively. (See, e.g., Wigler, M. et al. (1977) Cell 11:223-232; and Lowy, I. et al. (1980) Cell 22:817-823) Also, antimetabolite, antibiotic, or herbicide resistance can be used as the basis for selection. For example, dhfr confers resistance to methotrexate; npt confers resistance to the aminoglycosides neomycin and G-418; and als or pat confer resistance to chlorsulfuron and phosphinotricin acetyltransferase, respectively. (See, e.g., Wigler, M. et al. (1980) Proc. Natl. Acad. Sci. USA 77:3567-3570; Colbere-Garapin, F. et al (1981) J. Mol. Biol. 150:1-14; and Murry, supra.) Additional selectable genes have been described, e.g., trpB, which allows cells to utilize indole in place of tryptophan, or hisD, which allows cells to utilize histinol in place of histidine. (See, e.g., Hartman, S. C. and R. C. Mulligan (1988) Proc. Natl. Acad. Sci. USA 85:8047-8051.) Visible markers, e.g., anthocyanins, β glucuronidase and its substrate GUS, luciferase and its substrate luciferin may be used. Green fluorescent proteins (GFP) (Clontech, Palo Alto, Calif.) can also be used. These markers can be used not only to identify transformants, but also to quantify the amount of transient or stable protein expression attributable to a specific vector system. (See, e.g., Rhodes, C. A. et al. (1995) Methods Mol. Biol. 55:121-131.)

Although the presence/absence of marker gene expression suggests that the gene of interest is also present, the presence and expression of the gene may need to be confirmed. For example, if the sequence encoding MSP is inserted within a marker gene sequence, transformed cells containing sequences encoding MSP can be identified by the absence of marker gene function. Alternatively, a marker gene can be placed in tandem with a sequence encoding MSP under the control of a single promoter. Expression of the marker gene in response to induction or selection usually indicates expression of the tandem gene as well.

Alternatively, host cells which contain the nucleic acid sequence encoding MSP and express MSP may be identified by a variety of procedures known to those of skill in the art. These procedures include, but are not limited to, DNA-DNA or DNA-RNA hybridizations and protein bioassay or immunoassay techniques which include membrane, solution, or chip based technologies for the detection and/or quantification of nucleic acid or protein sequences.

The presence of polynucleotide sequences encoding MSP can be detected by DNA-DNA or DNA-RNA hybridization or amplification using probes or fragments or fragments of polynucleotides encoding MSP. Nucleic acid amplification based assays involve the use of oligonucleotides or oligomers based on the sequences encoding MSP to detect transformants containing DNA or RNA encoding MSP.

A variety of protocols for detecting and measuring the expression of MSP, using either polyclonal or monoclonal antibodies specific for the protein, are known in the art. Examples of such techniques include enzyme-linked immunosorbent assays (ELISAs), radioimmunoassays (RIAs), and fluorescence activated cell sorting (FACS). A two-site, monoclonal-based immunoassay utilizing monoclonal antibodies reactive to two non-interfering epitopes on MSP is preferred, but a competitive binding assay may be employed. These and other assays are well described in the art. (See, e.g., Hampton, R. et al. (1990) Serological Methods, a Laboratory Manual, APS Press, St. Paul Minn., Section IV; and Maddox, D. E. et al. (1983) J. Exp. Med. 158:1211-1216).

A wide variety of labels and conjugation techniques are known by those skilled in the art and may be used in various nucleic acid and amino acid assays. Means for producing labeled hybridization or PCR probes for detecting sequences related to polynucleotides encoding MSP include oligolabeling, nick translation, end-labeling, or PCR amplification using a labeled nucleotide. Alternatively, the sequences encoding MSP, or any fragments thereof, may be cloned into a vector for the production of an mRNA probe. Such vectors are known in the art, are commercially available, and may be used to synthesize RNA probes in vitro by addition of an appropriate RNA polymerase such as T7, T3, or SP6 and labeled nucleotides. These procedures may be conducted using a variety of commercially available kits, such as those provided by Pharmacia & Upjohn (Kalamazoo, Mich.), Promega (Madison, Wis.), and U.S. Biochemical Corp. (Cleveland, Ohio). Suitable reporter molecules or labels which may be used for ease of detection include radionuclides, enzymes, fluorescent, chemiluminescent, or chromogenic agents, as well as substrates, cofactors, inhibitors, magnetic particles, and the like.

Host cells transformed with nucleotide sequences encoding MSP may be cultured under conditions suitable for the expression and recovery of the protein from cell culture. The protein produced by a transformed cell may be secreted or contained intracellularly depending on the sequence and/or the vector used. As will be understood by those of skill in the art, expression vectors containing polynucleotides which encode MSP may be designed to contain signal sequences which direct secretion of MSP through a prokaryotic or eukaryotic cell membrane. Other constructions may be used to join sequences encoding MSP to nucleotide sequences encoding a polypeptide domain which will facilitate purification of soluble proteins. Such purification facilitating domains include, but are not limited to, metal chelating peptides such as histidine-tryptophan modules that allow purification on immobilized metals, protein A domains that allow purification on immobilized immunoglobulin, and the domain utilized in the FLAGS extension/affinity purification system (Immunex Corp., Seattle, Wash.). The inclusion of cleavable linker sequences, such as those specific for Factor XA or enterokinase (Invitrogen, San Diego, Calif.), between the purification domain and the MSP encoding sequence may be used to facilitate purification. One such expression vector provides for expression of a fusion protein containing MSP and a nucleic acid encoding 6 histidine residues preceding a thioredoxin or an enterokinase cleavage site. The histidine residues facilitate purification on immobilized metal ion affinity chromatography (IMAC). (See, e.g., Porath, J. et al. (1992) Prot. Exp. Purif. 3:263-281.) The enterokinase cleavage site provides a means for purifying MSP from the fusion protein. (See, e.g., Kroll, D. J. et al. (1993) DNA Cell Biol. 12:441-453.)

Fragments of MSP may be produced not only by recombinant production, but also by direct peptide synthesis using solid-phase techniques. (See, e.g., Creighton, T. E. (1984) Protein: Structures and Molecular Properties, pp. 55-60, W.H. Freeman and Co., New York N.Y.) Protein synthesis may be performed by manual techniques or by automation. Automated synthesis may be achieved, for example, using the Applied Biosystems 431A Peptide Synthesizer (Perkin Elmer). Various fragments of MSP may be synthesized separately and then combined to produce the full length molecule.

THERAPEUTICS

Chemical and structural homology exits among the human membrane spanning proteins of the invention. In addition, the expression of MSP is closely associated with cell proliferation, and with tissues associated cancer, the immune response, and in reproductive tissues. Therefore, MSP appears to play a role in cancer, immunological, and reproductive disorders, in particular where increased activity or synthesis appears to be associated with these disorders.

In one embodiment, antagonists which decrease the expression or activity of MSP may be administered to a subject to treat or prevent a neoplastic disorder such as adenocarcinoma, leukemia, lymphoma, melanoma, myeloma, sarcoma, and teratocarcinoma. Such cancers include, but are not limited to, cancers of the adrenal gland, bladder, bone, bone marrow, brain, breast, cervix, gall bladder, ganglia, gastrointestinal tract, heart, kidney, liver, lung, muscle, ovary, pancreas, parathyroid, penis, prostate, salivary glands, skin, spleen, testis, thymus, thyroid, and uterus. In one aspect, antibodies which specifically bind MSP may be used directly as an antagonist or indirectly as a targeting or delivery mechanism for bringing a pharmaceutical agent to cells or tissue which express MSP.

In another embodiment, a vector expressing the complement of the polynucleotide encoding MSP may be administered to a subject to treat or prevent a neoplastic disorder including, but not limited to, those listed above.

In yet another embodiment, antagonists which decrease the activity of MSP may be administered to a subject to treat or prevent an immunological disorder. Such immunological disorders may be associated with conditions such as AIDS, Addison's disease, adult respiratory distress syndrome, allergies, anemia, asthma, atherosclerosis, bronchitis, cholecystitus, Crohn's disease, ulcerative colitis, atopic dermatitis, dermatomyositis, diabetes mellitus, emphysema, atrophic gastritis, glomerulonephritis, gout, Graves' disease, hypereosinophilia, irritable bowel syndrome, lupus erythematosus, multiple sclerosis, myasthenia gravis, myocardial or pericardial inflammation, osteoarthritis, osteoporosis, pancreatitis, polymyositis, rheumatoid arthritis, scleroderma, Sjögren's syndrome, and autoimmune thyroiditis; complications of cancer, hemodialysis, extracorporeal circulation; viral, bacterial, fungal, parasitic, protozoal, and helminthic infections; and trauma. In one aspect, antibodies which specifically bind MSP may be used directly as an antagonist or indirectly as a targeting or delivery mechanism for bringing a pharmaceutical agent to cells or tissue which express MSP.

In another embodiment, a vector expressing the complement of the polynucleotide encoding MSP may be administered to a subject to treat or prevent an immunological disorder including, but not limited to, those listed above.

In a further embodiment, an antagonist of MSP may be administered to a subject to treat or prevent a reproductive disorder. Such a reproductive disorder may include, but is not limited to, disorders of prolactin production; infertility, including tubal disease, ovulatory defects, and endometriosis; disruptions of the estrous cycle, disruptions of the menstrual cycle, polycystic ovary syndrome, ovarian hyperstimulation syndrome, endometrial and ovarian tumors, autoimmune disorders, ectopic pregnancy, and teratogenesis; cancer of the breast, uterine fibroids, fibrocystic breast disease, galactorrhea; disruptions of spermatogenesis, abnormal sperm physiology, cancer of the testis, cancer of the prostate, benign prostatic hyperplasia, prostatitis, Peyronie's disease, carcinoma of the male breast and gynecomastia. In one aspect, an antibody which specifically binds MSP may be used directly as an antagonist or indirectly as a targeting or delivery mechanism for bringing a pharmaceutical agent to cells or tissue which express MSP.

In an additional embodiment, a vector expressing the complement of the polynucleotide encoding MSP may be administered to a subject to treat or prevent a reproductive disorder including, but not limited to, those described above.

In other embodiments, any of the proteins, antagonists, antibodies, agonists, complementary sequences, or vectors of the invention may be administered in combination with other appropriate therapeutic agents. Selection of the appropriate agents for use in combination therapy may be made by one of ordinary skill in the art, according to conventional pharmaceutical principles. The combination of therapeutic agents may act synergistically to effect the treatment or prevention of the various disorders described above. Using this approach, one may be able to achieve therapeutic efficacy with lower dosages of each agent, thus reducing the potential for adverse side effects.

An antagonist of MSP may be produced using methods which are generally known in the art. In particular, purified MSP may be used to produce antibodies or to screen libraries of pharmaceutical agents to identify those which specifically bind MSP. Antibodies to MSP may also be generated using methods that are well known in the art. Such antibodies may include, but are not limited to, polyclonal, monoclonal, chimeric, and single chain antibodies, Fab fragments, and fragments produced by a Fab expression library. Neutralizing antibodies (i.e., those which inhibit dimer formation) are especially preferred for therapeutic use.

For the production of antibodies, various hosts including goats, rabbits, rats, mice, humans, and others may be immunized by injection with MSP or with any fragment or oligopeptide thereof which has immunogenic properties. Depending on the host species, various adjuvants may be used to increase immunological response. Such adjuvants include, but are not limited to, Freund's, mineral gels such as aluminum hydroxide, and surface active substances such as lysolecithin, pluronic polyols, polyanions, peptides, oil emulsions, KLH, and dinitrophenol. Among adjuvants used in humans, BCG (bacilli Calmette-Guerin) and Corynebacterium parvum are especially preferable.

It is preferred that the oligopeptides, peptides, or fragments used to induce antibodies to MSP have an amino acid sequence consisting of at least about 5 amino acids, and, more preferably, of at least about 10 amino acids. It is also preferable that these oligopeptides, peptides, or fragments are identical to a portion of the amino acid sequence of the natural protein and contain the entire amino acid sequence of a small, naturally occurring molecule. Short stretches of MSP amino acids may be fused with those of another protein, such as KLH, and antibodies to the chimeric molecule may be produced.

Monoclonal antibodies to MSP may be prepared using any technique which provides for the production of antibody molecules by continuous cell lines in culture. These include, but are not limited to, the hybridoma technique, the human B-cell hybridoma technique, and the EBV-hybridoma technique. (See, e.g., Kohler, G. et al. (1975) Nature 256:495-497; Kozbor, D. et al. (1985) J. Immunol. Methods 81:31-42; Cote, R. J. et al. (1983) Proc. Natl. Acad. Sci. USA 80:2026-2030; and Cole, S. P. et al. (1984) Mol. Cell Biol. 62:109-120.)

In addition, techniques developed for the production of “chimeric antibodies,” such as the splicing of mouse antibody genes to human antibody genes to obtain a molecule with appropriate antigen specificity and biological activity, can be used. (See, e.g., Morrison, S. L. et al. (1984) Proc. Natl. Acad. Sci. USA 81:6851-6855; Neuberger, M. S. et al. (1984) Nature 312:604-608; and Takeda, S. et al. (1985) Nature 314:452-454.) Alternatively, techniques described for the production of single chain antibodies may be adapted, using methods known in the art, to produce MSP-specific single chain antibodies. Antibodies with related specificity, but of distinct idiotypic composition, may be generated by chain shuffling from random combinatorial immunoglobulin libraries. (See, e.g., Burton, D. R. (1991) Proc. Natl. Acad. Sci. USA 88:10134-10137.)

Antibodies may also be produced by inducing in vivo production in the lymphocyte population or by screening immunoglobulin libraries or panels of highly specific binding reagents as disclosed in the literature. (See, e.g., Orlandi, R. et al. (1989) Proc. Natl. Acad. Sci. USA 86:3833-3837; and Winter, G. et al. (1991) Nature 349:293-299.)

Antibody fragments which contain specific binding sites for MSP may also be generated. For example, such fragments include, but are not limited to, F(ab′)2 fragments produced by pepsin digestion of the antibody molecule and Fab fragments generated by reducing the disulfide bridges of the F(ab′)2 fragments. Alternatively, Fab expression libraries may be constructed to allow rapid and easy identification of monoclonal Fab fragments with the desired specificity. (See, e.g., Huse, W. D. et al. (1989) Science 246:1275-1281.)

Various immunoassays may be used for screening to identify antibodies having the desired specificity. Numerous protocols for competitive binding or immunoradiometric assays using either polyclonal or monoclonal antibodies with established specificities are well known in the art. Such immunoassays typically involve the measurement of complex formation between MSP and its specific antibody. A two-site, monoclonal-based immunoassay utilizing monoclonal antibodies reactive to two non-interfering MSP epitopes is preferred, but a competitive binding assay may also be employed. (Maddox, supra.)

In another embodiment of the invention, the polynucleotides encoding MSP, or any fragment or complement thereof, may be used for therapeutic purposes. In one aspect, the complement of the polynucleotide encoding MSP may be used in situations in which it would be desirable to block the transcription of the mRNA. In particular, cells may be transformed with sequences complementary to polynucleotides encoding MSP. Thus, complementary molecules or fragments may be used to modulate MSP activity, or to achieve regulation of gene function. Such technology is now well known in the art, and sense or antisense oligonucleotides or larger fragments can be designed from various locations along the coding or control regions of sequences encoding MSP.

Expression vectors derived from retroviruses, adenoviruses, or herpes or vaccinia viruses, or from various bacterial plasmids, may be used for delivery of nucleotide sequences to the targeted organ, tissue, or cell population. Methods which are well known to those skilled in the art can be used to construct vectors which will express nucleic acid sequences complementary to the polynucleotides of the gene encoding MSP. (See, e.g., Sambrook, supra; and Ausubel, supra.)

Genes encoding MSP can be turned off by transforming a cell or tissue with expression vectors which express high levels of a polynucleotide, or fragment thereof, encoding MSP. Such constructs may be used to introduce untranslatable sense or antisense sequences into a cell. Even in the absence of integration into the DNA, such vectors may continue to transcribe RNA molecules until they are disabled by endogenous nucleases. Transient expression may last for a month or more with a non-replicating vector, and may last even longer if appropriate replication elements are part of the vector system.

As mentioned above, modifications of gene expression can be obtained by designing complementary sequences or antisense molecules (DNA, RNA, or PNA) to the control, 5′, or regulatory regions of the gene encoding MSP. Oligonucleotides derived from the transcription initiation site, e.g., between about positions −10 and +10 from the start site, are preferred. Similarly, inhibition can be achieved using triple helix base-pairing methodology. Triple helix pairing is useful because it causes inhibition of the ability of the double helix to open sufficiently for the binding of polymerases, transcription factors, or regulatory molecules. Recent therapeutic advances using triplex DNA have been described in the literature. (See, e.g., Gee, J. E. et al. (1994) in Huber, B. E. and B. I. Carr, Molecular and Immunologic Approaches, Futura Publishing Co., Mt. Kisco N.Y., pp. 163-177.) A complementary sequence or antisense molecule may also be designed to block translation of mRNA by preventing the transcript from binding to ribosomes.

Ribozymes, enzymatic RNA molecules, may also be used to catalyze the specific cleavage of RNA. The mechanism of ribozyme action involves sequence-specific hybridization of the ribozyme molecule to complementary target RNA, followed by endonucleolytic cleavage. For example, engineered hammerhead motif ribozyme molecules may specifically and efficiently catalyze endonucleolytic cleavage of sequences encoding MSP.

Specific ribozyme cleavage sites within any potential RNA target are initially identified by scanning the target molecule for ribozyme cleavage sites, including the following sequences: GUA, GUU, and GUC. Once identified, short RNA sequences of between 15 and 20 ribonucleotides, corresponding to the region of the target gene containing the cleavage site, may be evaluated for secondary structural features which may render the oligonucleotide inoperable. The suitability of candidate targets may also be evaluated by testing accessibility to hybridization with complementary oligonucleotides using ribonuclease protection assays.

Complementary ribonucleic acid molecules and ribozymes of the invention may be prepared by any method known in the art for the synthesis of nucleic acid molecules. These include techniques for chemically synthesizing oligonucleotides such as solid phase phosphoramidite chemical synthesis. Alternatively, RNA molecules may be generated by in vitro and in vivo transcription of DNA sequences encoding MSP. Such DNA sequences may be incorporated into a wide variety of vectors with suitable RNA polymerase promoters such as T7 or SP6. Alternatively, these cDNA constructs that synthesize complementary RNA, constitutively or inducibly, can be introduced into cell lines, cells, or tissues.

RNA molecules may be modified to increase intracellular stability and half-life. Possible modifications include, but are not limited to, the addition of flanking sequences at the 5′ and/or 3′ ends of the molecule, or the use of phosphorothioate or 2′ O-methyl rather than phosphodiesterase linkages within the backbone of the molecule. This concept is inherent in the production of PNAs and can be extended in all of these molecules by the inclusion of nontraditional bases such as inosine, queosine, and wybutosine, as well as acetyl-, methyl-, thio-, and similarly modified forms of adenine, cytidine, guanine, thymine, and uridine which are not as easily recognized by endogenous endonucleases.

Many methods for introducing vectors into cells or tissues are available and equally suitable for use in vivo, in vitro, and ex vivo. For ex vivo therapy, vectors may be introduced into stem cells taken from the patient and clonally propagated for autologous transplant back into that same patient. Delivery by transfection, by liposome injections, or by polycationic amino polymers may be achieved using methods which are well known in the art. (See, e.g., Goldman, C. K. et al. (1997) Nat. Biotechnol. 15:462-466.)

Any of the therapeutic methods described above may be applied to any subject in need of such therapy, including, for example, mammals such as dogs, cats, cows, horses, rabbits, monkeys, and most preferably, humans.

An additional embodiment of the invention relates to the administration of a pharmaceutical or sterile composition, in conjunction with a pharmaceutically acceptable carrier, for any of the therapeutic effects discussed above. Such pharmaceutical compositions may consist of MSP, antibodies to MSP, and mimetics, agonists, antagonists, or inhibitors of MSP. The compositions may be administered alone or in combination with at least one other agent, such as a stabilizing compound, which may be administered in any sterile, biocompatible pharmaceutical carrier including, but not limited to, saline, buffered saline, dextrose, and water. The compositions may be administered to a patient alone, or in combination with other agents, drugs, or hormones.

The pharmaceutical compositions utilized in this invention may be administered by any number of routes including, but not limited to, oral, intravenous, intramuscular, intra-arterial, intramedullary, intrathecal, intraventricular, transdermal, subcutaneous, intraperitoneal, intranasal, enteral, topical, sublingual, or rectal means.

In addition to the active ingredients, these pharmaceutical compositions may contain suitable pharmaceutically-acceptable carriers comprising excipients and auxiliaries which facilitate processing of the active compounds into preparations which can be used pharmaceutically. Further details on techniques for formulation and administration may be found in the latest edition of Remington's Pharmaceutical Sciences (Maack Publishing Co., Easton Pa.).

Pharmaceutical compositions for oral administration can be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions, and the like, for ingestion by the patient.

Pharmaceutical preparations for oral use can be obtained through combining active compounds with solid excipient and processing the resultant mixture of granules (optionally, after grinding) to obtain tablets or dragee cores. Suitable auxiliaries can be added, if desired. Suitable excipients include carbohydrate or protein fillers, such as sugars, including lactose, sucrose, mannitol, and sorbitol; starch from corn, wheat, rice, potato, or other plants; cellulose, such as methyl cellulose, hydroxypropylmethyl-cellulose, or sodium carboxymethylcellulose; gums, including arabic and tragacanth; and proteins, such as gelatin and collagen. If desired, disintegrating or solubilizing agents may be added, such as the cross-linked polyvinyl pyrrolidone, agar, and alginic acid or a salt thereof, such as sodium alginate.

Dragee cores may be used in conjunction with suitable coatings, such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound, i.e., dosage.

Pharmaceutical preparations which can be used orally include push-fit capsules made of gelatin, as well as soft, sealed capsules made of gelatin and a coating, such as glycerol or sorbitol. Push-fit capsules can contain active ingredients mixed with fillers or binders, such as lactose or starches, lubricants, such as talc or magnesium stearate, and, optionally, stabilizers. In soft capsules, the active compounds may be dissolved or suspended in suitable liquids, such as fatty oils, liquid, or liquid polyethylene glycol with or without stabilizers.

Pharmaceutical formulations suitable for parenteral administration may be formulated in aqueous solutions, preferably in physiologically compatible buffers such as Hanks's solution, Ringer's solution, or physiologically buffered saline. Aqueous injection suspensions may contain substances which increase the viscosity of the suspension, such as sodium carboxymethyl cellulose, sorbitol, or dextran. Additionally, suspensions of the active compounds may be prepared as appropriate oily injection suspensions. Suitable lipophilic solvents or vehicles include fatty oils, such as sesame oil, or synthetic fatty acid esters, such as ethyl oleate, triglycerides, or liposomes. Non-lipid polycationic amino polymers may also be used for delivery. Optionally, the suspension may also contain suitable stabilizers or agents to increase the solubility of the compounds and allow for the preparation of highly concentrated solutions.

For topical or nasal administration, penetrants appropriate to the particular barrier to be permeated are used in the formulation. Such penetrants are generally known in the art.

The pharmaceutical compositions of the present invention may be manufactured in a manner that is known in the art, e.g., by means of conventional mixing, dissolving, granulating, dragee-making, levigating, emulsifying, encapsulating, entrapping, or lyophilizing processes.

The pharmaceutical composition may be provided as a salt and can be formed with many acids, including but not limited to, hydrochloric, sulfuric, acetic, lactic, tartaric, malic, and succinic acid. Salts tend to be more soluble in aqueous or other protonic solvents than are the corresponding free base forms. In other cases, the preferred preparation may be a lyophilized powder which may contain any or all of the following: 1 mM to 50 mM histidine, 0.1% to 2% sucrose, and 2% to 7% mannitol, at a pH range of 4.5 to 5.5, that is combined with buffer prior to use.

After pharmaceutical compositions have been prepared, they can be placed in an appropriate container and labeled for treatment of an indicated condition. For administration of MSP, such labeling would include amount, frequency, and method of administration.

Pharmaceutical compositions suitable for use in the invention include compositions wherein the active ingredients are contained in an effective amount to achieve the intended purpose. The determination of an effective dose is well within the capability of those skilled in the art.

For any compound, the therapeutically effective dose can be estimated initially either in cell culture assays, e.g., of neoplastic cells or in animal models such as mice, rats, rabbits, dogs, or pigs. An animal model may also be used to determine the appropriate concentration range and route of administration. Such information can then be used to determine useful doses and routes for administration in humans.

A therapeutically effective dose refers to that amount of active ingredient, for example MSP or fragments thereof, antibodies of MSP, and agonists, antagonists or inhibitors of MSP, which ameliorates the symptoms or condition. Therapeutic efficacy and toxicity may be determined by standard pharmaceutical procedures in cell cultures or with experimental animals, such as by calculating the ED50 (the dose therapeutically effective in 50% of the population) or LD50 (the dose lethal to 50% of the population) statistics. The dose ratio of therapeutic to toxic effects is the therapeutic index, and it can be expressed as the ED50/LD50 ratio. Pharmaceutical compositions which exhibit large therapeutic indices are preferred. The data obtained from cell culture assays and animal studies are used to formulate a range of dosage for human use. The dosage contained in such compositions is preferably within a range of circulating concentrations that includes the ED50 with little or no toxicity. The dosage varies within this range depending upon the dosage form employed, the sensitivity of the patient, and the route of administration.

The exact dosage will be determined by the practitioner, in light of factors related to the subject requiring treatment. Dosage and administration are adjusted to provide sufficient levels of the active moiety or to maintain the desired effect. Factors which may be taken into account include the severity of the disease state, the general health of the subject, the age, weight, and gender of the subject, time and frequency of administration, drug combination(s), reaction sensitivities, and response to therapy. Long-acting pharmaceutical compositions may be administered every 3 to 4 days, every week, or biweekly depending on the half-life and clearance rate of the particular formulation.

Normal dosage amounts may vary from about 0.1 μg to 100,000 μg, up to a total dose of about 1 gram, depending upon the route of administration. Guidance as to particular dosages and methods of delivery is provided in the literature and generally available to practitioners in the art. Those skilled in the art will employ different formulations for nucleotides than for proteins or their inhibitors. Similarly, delivery of polynucleotides or polypeptides will be specific to particular cells, conditions, locations, etc.

DIAGNOSTICS

In another embodiment, antibodies which specifically bind MSP may be used for the diagnosis of disorders characterized by expression of MSP, or in assays to monitor patients being treated with MSP or agonists, antagonists, or inhibitors of MSP. Antibodies useful for diagnostic purposes may be prepared in the same manner as described above for therapeutics. Diagnostic assays for MSP include methods which utilize the antibody and a label to detect MSP in human body fluids or in extracts of cells or tissues. The antibodies may be used with or without modification, and may be labeled by covalent or non-covalent attachment of a reporter molecule. A wide variety of reporter molecules, several of which are described above, are known in the art and may be used.

A variety of protocols for measuring MSP, including ELISAs, RIAs, and FACS, are known in the art and provide a basis for diagnosing altered or abnormal levels of MSP expression. Normal or standard values for MSP expression are established by combining body fluids or cell extracts taken from normal mammalian subjects, preferably human, with antibody to MSP under conditions suitable for complex formation The amount of standard complex formation may be quantitated by various methods, preferably by photometric means. Quantities of MSP expressed in subject, control, and disease samples from biopsied tissues are compared with the standard values. Deviation between standard and subject values establishes the parameters for diagnosing disease.

In another embodiment of the invention, the polynucleotides encoding MSP may be used for diagnostic purposes. The polynucleotides which may be used include oligonucleotide sequences, complementary RNA and DNA molecules, and PNAs. The polynucleotides may be used to detect and quantitate gene expression in biopsied tissues in which expression of MSP may be correlated with disease. The diagnostic assay may be used to determine absence, presence, and excess expression of MSP, and to monitor regulation of MSP levels during therapeutic intervention.

In one aspect, hybridization with PCR probes which are capable of detecting polynucleotide sequences, including genomic sequences, encoding MSP or closely related molecules may be used to identify nucleic acid sequences which encode MSP. The specificity of the probe, whether it is made from a highly specific region, e.g., the 5′ regulatory region, or from a less specific region, e.g., a conserved motif, and the stringency of the hybridization or amplification (maximal, high, intermediate, or low), will determine whether the probe identifies only naturally occurring sequences encoding MSP, alleles, or related sequences.

Probes may also be used for the detection of related sequences, and should preferably have at least 50% sequence identity to any of the MSP encoding sequences. The hybridization probes of the subject invention may be DNA or RNA and derived from the nucleotide sequence of SEQ ID NO:18 through SEQ ID NO:34, or from genomic sequences including promoters, enhancers, and introns of the MSP gene.

Means for producing specific hybridization probes for DNAs encoding MSP include the cloning of polynucleotide sequences encoding MSP or MSP derivatives into vectors for the production of mRNA probes. Such vectors are known in the art, are commercially available, and may be used to synthesize RNA probes in vitro by means of the addition of the appropriate RNA polymerases and the appropriate labeled nucleotides. Hybridization probes may be labeled by a variety of reporter groups, for example, by radionuclides such as ³²P or ³⁵S, or by enzymatic labels, such as alkaline phosphatase coupled to the probe via avidin/biotin coupling systems, and the like.

Polynucleotide sequences encoding MSP may be used for the diagnosis of a disorder associated with expression of MSP. Examples of such a disorder include, but are not limited to, a neoplastic disorder such as adenocarcinoma, leukemia, lymphoma, melanoma, myeloma, sarcoma, and teratocarcinoma. Such cancers include, but are not limited to, cancers of the adrenal gland, bladder, bone, bone marrow, brain, breast, cervix, gall bladder, ganglia, gastrointestinal tract, heart, kidney, liver, lung, muscle, ovary, pancreas, parathyroid, penis, prostate, salivary glands, skin, spleen, testis, thymus, thyroid, and uterus; an immunological disorder such as AIDS, Addison's disease, adult respiratory distress syndrome, allergies, anemia, asthma, atherosclerosis, bronchitis, cholecystitus, Crohn's disease, ulcerative colitis, atopic dermatitis, dermatomyositis, diabetes mellitus, emphysema, atrophic gastritis, glomerulonephritis, gout, Graves' disease, hypereosinophilia, irritable bowel syndrome, lupus erythematosus, multiple sclerosis, myasthenia gravis, myocardial or pericardial inflammation, osteoarthritis, osteoporosis, pancreatitis, polymyositis, rheumatoid arthritis, scleroderma, Sjögren's syndrome, and autoimmune thyroiditis; complications of cancer, hemodialysis, extracorporeal circulation; viral, bacterial, fungal, parasitic, protozoal, and helminthic infections; and trauma; a reproductive disorder such as, disorders of prolactin production; infertility, including tubal disease, ovulatory defects, and endometriosis; disruptions of the estrous cycle, disruptions of the menstrual cycle, polycystic ovary syndrome, ovarian hyperstimulation syndrome, endometrial and ovarian tumors, autoimmune disorders, ectopic pregnancy, and teratogenesis; cancer of the breast, uterine fibroids, fibrocystic breast disease, galactorrhea; disruptions of spermatogenesis, abnormal sperm physiology, cancer of the testis, cancer of the prostate, benign prostatic hyperplasia, prostatitis, Peyronie's disease, carcinoma of the male breast and gynecomastia. The polynucleotide sequences encoding MSP may be used in Southern or northern analysis, dot blot, or other membrane-based technologies; in PCR technologies; in dipstick, pin, and ELISA assays; and in microarrays utilizing fluids or tissues from patients to detect altered MSP expression. Such qualitative or quantitative methods are well known in the art.

In a particular aspect, the nucleotide sequences encoding MSP may be useful in assays that detect the presence of associated disorders, particularly those mentioned above. The nucleotide sequences encoding MSP may be labeled by standard methods and added to a fluid or tissue sample from a patient under conditions suitable for the formation of hybridization complexes. After a suitable incubation period, the sample is washed and the signal is quantitated and compared with a standard value. If the amount of signal in the patient sample is significantly altered in comparison to a control sample then the presence of altered levels of nucleotide sequences encoding MSP in the sample indicates the presence of the associated disorder. Such assays may also be used to evaluate the efficacy of a particular therapeutic treatment regimen in animal studies, in clinical trials, or to monitor the treatment of an individual patient.

In order to provide a basis for the diagnosis of a disorder associated with expression of MSP, a normal or standard profile for expression is established. This may be accomplished by combining body fluids or cell extracts taken from normal subjects, either animal or human, with a sequence, or a fragment thereof, encoding MSP, under conditions suitable for hybridization or amplification. Standard hybridization may be quantified by comparing the values obtained from normal subjects with values from an experiment in which a known amount of a substantially purified polynucleotide is used. Standard values obtained in this manner may be compared with values obtained from samples from patients who are symptomatic for a disorder. Deviation from standard values is used to establish the presence of a disorder.

Once the presence of a disorder is established and a treatment protocol is initiated, hybridization assays may be repeated on a regular basis to determine if the level of expression in the patient begins to approximate that which is observed in the normal subject. The results obtained from successive assays may be used to show the efficacy of treatment over a period ranging from several days to months.

With respect to cancer, the presence of a relatively high amount of transcript in biopsied tissue from an individual may indicate a predisposition for the development of the disease, or may provide a means for detecting the disease prior to the appearance of actual clinical symptoms. A more definitive diagnosis of this type may allow health professionals to employ preventative measures or aggressive treatment earlier thereby preventing the development or further progression of the cancer.

Additional diagnostic uses for oligonucleotides designed from the sequences encoding MSP may involve the use of PCR. These oligomers may be chemically synthesized, generated enzymatically, or produced in vitro. Oligomers will preferably contain a fragment of a polynucleotide encoding MSP, or a fragment of a polynucleotide complementary to the polynucleotide encoding MSP, and will be employed under optimized conditions for identification of a specific gene or condition. Oligomers may also be employed under less stringent conditions for detection or quantitation of closely related DNA or RNA sequences.

Methods which may also be used to quantitate the expression of MSP include radiolabeling or biotinylating nucleotides, coamplification of a control nucleic acid, and interpolating results from standard curves. (See, e.g., Melby, P. C. et al. (1993) J. Immunol. Methods 159:235-244; and Duplaa, C. et al. (1993) Anal. Biochem. 212:229-236.) The speed of quantitation of multiple samples may be accelerated by running the assay in an ELISA format where the oligomer of interest is presented in various dilutions and a spectrophotometric or colorimetric response gives rapid quantitation.

In further embodiments, oligonucleotides or longer fragments derived from any of the polynucleotide sequences described herein may be used as targets in a microarray. The microarray can be used to monitor the expression level of large numbers of genes simultaneously and to identify genetic variants, mutations, and polymorphisms. This information may be used to determine gene function, to understand the genetic basis of a disorder, to diagnose a disorder, and to develop and monitor the activities of therapeutic agents.

Microarrays may be prepared, used, and analyzed using methods known in the art. (See, e.g., Brennan, T. M. et al. (1995) U.S. Pat. No. 5,474,796; Schena, M. et al. (1996) Proc. Natl. Acad. Sci. USA 93:10614-10619; Baldeschweiler et al. (1995) PCT application WO95/251116; Shalon, D. et al. (1995) PCT application WO95/35505; Heller, R. A. et al. (1997) Proc. Natl. Acad. Sci. USA 94:2150-2155; and Heller, M. J. et al. (1997) U.S. Pat. No. 5,605,662.)

In another embodiment of the invention, nucleic acid sequences encoding MSP may be used to generate hybridization probes useful in mapping the naturally occurring genomic sequence. The sequences may be mapped to a particular chromosome, to a specific region of a chromosome, or to artificial chromosome constructions, e.g., human artificial chromosomes (HACs), yeast artificial chromosomes (YACs), bacterial artificial chromosomes (BACs), bacterial P1 constructions, or single chromosome cDNA libraries. (See, e.g., Price, C. M. (1993) Blood Rev. 7:127-134; and Trask, B. J. (1991) Trends Genet. 7:149-154.)

Fluorescent in situ hybridization (FISH) may be correlated with other physical chromosome mapping techniques and genetic map data. (See, e.g., Heinz-Ulrich, et al. (1995) in Meyers, R. A. (ed.) Molecular Biology and Biotechnology, VCH Publishers, New York N.Y., pp. 965-968.) Examples of genetic map data can be found in various scientific journals or at the Online Mendelian Inheritance in Man (OMIM) site. Correlation between the location of the gene encoding MSP on a physical chromosomal map and a specific disorder, or a predisposition to a specific disorder, may help define the region of DNA associated with that disorder. The nucleotide sequences of the invention may be used to detect differences in gene sequences among normal, carrier, and affected individuals.

In situ hybridization of chromosomal preparations and physical mapping techniques, such as linkage analysis using established chromosomal markers, may be used for extending genetic maps. Often the placement of a gene on the chromosome of another mammalian species, such as mouse, may reveal associated markers even if the number or arm of a particular human chromosome is not known. New sequences can be assigned to chromosomal arms by physical mapping. This provides valuable information to investigators searching for disease genes using positional cloning or other gene discovery techniques. Once the disease or syndrome has been crudely localized by genetic linkage to a particular genomic region, e.g., AT to 11q22-23, any sequences mapping to that area may represent associated or regulatory genes for further investigation. (See, e.g., Gatti, R. A. et al. (1988) Nature 336:577-580.) The nucleotide sequence of the subject invention may also be used to detect differences in the chromosomal location due to translocation, inversion, etc., among normal, carrier, or affected individuals.

In another embodiment of the invention, MSP, its catalytic or immunogenic fragments, or oligopeptides thereof can be used for screening libraries of compounds in any of a variety of drug screening techniques. The fragment employed in such screening may be free in solution, affixed to a solid support, borne on a cell surface, or located intracellularly. The formation of binding complexes between MSP and the agent being tested may be measured.

Another technique for drug screening provides for high throughput screening of compounds having suitable binding affinity to the protein of interest. (See, e.g., Geysen, et al. (1984) PCT application WO84/03564.) In this method, large numbers of different small test compounds are synthesized on a solid substrate, such as plastic pins or some other surface. The test compounds are reacted with MSP, or fragments thereof, and washed. Bound MSP is then detected by methods well known in the art. Purified MSP can also be coated directly onto plates for use in the aforementioned drug screening techniques. Alternatively, non-neutralizing antibodies can be used to capture the peptide and immobilize it on a solid support.

In another embodiment, one may use competitive drug screening assays in which neutralizing antibodies capable of binding MSP specifically compete with a test compound for binding MSP. In this manner, antibodies can be used to detect the presence of any peptide which shares one or more antigenic determinants with MSP.

In additional embodiments, the nucleotide sequences which encode MSP may be used in any molecular biology techniques that have yet to be developed, provided the new techniques rely on properties of nucleotide sequences that are currently known, including, but not limited to, such properties as the triplet genetic code and specific base pair interactions.

The examples below are provided to illustrate the subject invention and are not included for the purpose of limiting the invention.

### EXAMPLES

I. COLNNOT22 cDNA Library Construction

The COLNNOT22 library was constructed from microscopically normal colon tissue excised from a 56-year-old Caucasian female during a resection of the small intestine. The patient was diagnosed with Crohn's disease involving the ileum and ileal-colonic anastomosis. Patient history included a cholecystectomy and breast lesions. Family history included atherosclerosis in a grandparent and functional disorder of the intestine in the patient's mother.

The frozen tissue was homogenized and lysed using a Brinkmann Homogenizer Polytron PT-3000 (Brinkmann Instruments, Westbury, N.J.) in guanidinium isothiocyanate solution. The lysate was centrifuged over a 5.7 M CsCl cushion using an Beckman SW28 rotor in a Beckman L8-70M Ultracentrifuge (Beckman Instruments) for 18 hours at 25,000 rpm at ambient temperature. The RNA was extracted with acid phenol pH 4.7, precipitated using 0.3 M sodium acetate and 2.5 volumes of ethanol, resuspended in RNAse-free water, and DNase treated at 37° C. RNA extraction and precipitation were repeated as before. The mRNA was then isolated using the Qiagen Oligotex kit (QIAGEN, Inc., Chatsworth, Calif.) and used to construct the cDNA library.

The mRNA was handled according to the recommended protocols in the SuperScript plasmid system (Catalog #18248-013, GIBCO-BRL). cDNA synthesis was initiated with a NotI-oligo d(T) primer. Double stranded cDNA was blunted, ligated to EcoRI adaptors, digested with NotI, fractionated on a Sepharose CL4B column (Catalog #275105-01; Pharmacia), and those cDNAs exceeding 400 bp were ligated into the NotI and EcoRI sites of the pINCY 1 vector (Incyte). The plasmid pINCY 1 was subsequently transformed into DH5α competent cells (Catalog #18258-012; GIBCO-BRL).

II Isolation and Sequencing of cDNA Clones

Plasmid DNA was released from the cells and purified using the REAL Prep 96 plasmid kit (Catalog #26173; QIAGEN, Inc.). The recommended protocol was employed except for the following changes: 1) the bacteria were cultured in 1 ml of sterile Terrific Broth (Catalog #22711, GIBCO-BRL) with carbenicillin at 25 mg/l and glycerol at 0.4%; 2) after inoculation, the cultures were incubated for 19 hours and at the end of incubation, the cells were lysed with 0.3 ml of lysis buffer; and 3) following isopropanol precipitation, the plasmid DNA pellet was resuspended in 0.1 ml of distilled water. After the last step in the protocol, samples were transferred to a 96-well block for storage at 4° C.

The cDNAs were sequenced by the method of Sanger et al. (1975, J. Mol. Biol. 94:441f), using a Hamilton Micro Lab 2200 (Hamilton, Reno, Nev.) in combination with Peltier Thermal Cyclers (PTC200 from MJ Research, Watertown, Mass.) and Applied Biosystems 377 DNA Sequencing Systems.

III. Homology Searching of cDNA Clones and Their Deduced Proteins

The nucleotide sequences and/or amino acid sequences of the Sequence Listing were used to query sequences in the GenBank, SwissProt, BLOCKS, and Pima II databases. These databases, which contain previously identified and annotated sequences, were searched for regions of homology using BLAST (Basic Local Alignment Search Tool). (See, e.g., Altschul, S. F. (1993) J. Mol. Evol 36:290-300; and Altschul, et al. (1990) J. Mol. Biol. 215:403-410.)

BLAST produced alignments of both nucleotide and amino acid sequences to determine sequence similarity. Because of the local nature of the alignments, BLAST was especially useful in determining exact matches or in identifying homologs which may be of prokaryotic (bacterial) or eukaryotic (animal, fungal, or plant) origin. Other algorithms could have been used when dealing with primary sequence patterns and secondary structure gap penalties. (See, e.g., Smith, T. et al. (1992) Protein Engineering 5:35-51.) The sequences disclosed in this application have lengths of at least 49 nucleotides and have no more than 12% uncalled bases (where N is recorded rather than A, C, G, or T).

The BLAST approach searched for matches between a query sequence and a database sequence. BLAST evaluated the statistical significance of any matches found, and reported only those matches that satisfy the user-selected threshold of significance. In this application, threshold was set at 10⁻²⁵ for nucleotides and 10⁻⁸ for peptides.

Incyte nucleotide sequences were searched against the GenBank databases for primate (pri), rodent (rod), and other mammalian sequences (mam), and deduced amino acid sequences from the same clones were then searched against GenBank functional protein databases, mammalian (mamp), vertebrate (vrtp), and eukaryote (eukp), for homology.

IV. Northern Analysis

Northern analysis is a laboratory technique used to detect the presence of a transcript of a gene and involves the hybridization of a labeled nucleotide sequence to a membrane on which RNAs from a particular cell type or tissue have been bound. (See, e.g., Sambrook, supra, ch. 7; and Ausubel, F. M. et al. supra, ch. 4 and 16.)

Analogous computer techniques applying BLAST are used to search for identical or related molecules in nucleotide databases such as GenBank or LIFESEQ database (Incyte Pharmaceuticals). This analysis is much faster than multiple membrane-based hybridizations. In addition, the sensitivity of the computer search can be modified to determine whether any particular match is categorized as exact or homologous.

The basis of the search is the product score, which is defined as:

$$\frac{\% sequence identity \times \% maximum BLAST score}{100}$$

The product score takes into account both the degree of similarity between two sequences and the length of the sequence match. For example, with a product score of 40, the match will be exact within a 1% to 2% error, and, with a product score of 70, the match will be exact. Homologous molecules are usually identified by selecting those which show product scores between 15 and 40, although lower scores may identify related molecules.

The results of northern analysis are reported as a list of libraries in which the transcript encoding MSP occurs. Abundance and percent abundance are also reported. Abundance directly reflects the number of times a particular transcript is represented in a cDNA library, and percent abundance is abundance divided by the total number of sequences examined in the cDNA library.

V. Extension of MSP Encoding Polynucleotides

The nucleic acid sequence of Incyte Clones 77138, 1381884, 1427590, 1457779, 1481261, 1487802, 1718830, 1737775, 1794154, 2027624, 2057213, 2073804, 2175401, 2741580, 2779610, 2879792, and 3231062 were used to design oligonucleotide primers for extending partial nucleotide sequences to full length. For each nucleic acid sequence, one primer was synthesized to initiate extension of an antisense polynucleotide, and the other was synthesized to initiate extension of a sense polynucleotide. Primers were used to facilitate the extension of the known sequence “outward” generating amplicons containing new unknown nucleotide sequence for the region of interest. The initial primers were designed from the cDNA using OLIGO 4.06 (National Biosciences, Plymouth, Minn.), or another appropriate program, to be about 22 to 30 nucleotides in length, to have a GC content of about 50% or more, and to anneal to the target sequence at temperatures of about 68° C. to about 72° C. Any stretch of nucleotides which would result in hairpin structures and primer-primer dimerizations was avoided.

Selected human cDNA libraries (GIBCO/BRL) were used to extend the sequence. If more than one extension is necessary or desired, additional sets of primers are designed to further extend the known region.

High fidelity amplification was obtained by following the instructions for the XL-PCR kit (Perkin Elmer) and thoroughly mixing the enzyme and reaction mix. PCR was performed using the Peltier Thermal Cycler (PTC200; M.J. Research, Watertown, Mass.), beginning with 40 pmol of each primer and the recommended concentrations of all other components of the kit, with the following parameters:

| | |
|---|---|
| Step 1 | 94° C. for 1 min (initial denaturation) |
| Step 2 | 65° C. for 1 min |
| Step 3 | 68° C. for 6 min |
| Step 4 | 94° C. for 15 sec |
| Step 5 | 65° C. for 1 min |
| Step 6 | 68° C. for 7 min |
| Step 7 | Repeat steps 4 through 6 for an additional 15 cycles |
| Step 8 | 94° C. for 15 sec |
| Step 9 | 65° C. for 1 min |
| Step 10 | 68° C. for 7:15 min |
| Step 11 | Repeat steps 8 through 10 for an additional 12 cycles |
| Step 12 | 72° C. for 8 min |
| Step 13 | 4° C. (and holding) |

A 5 μl to 10 μl aliquot of the reaction mixture was analyzed by electrophoresis on a low concentration (about 0.6% to 0.8%) agarose mini-gel to determine which reactions were successful in extending the sequence. Bands thought to contain the largest products were excised from the gel, purified using QIAQUICK (QIAGEN Inc., Chatsworth, Calif.), and trimmed of overhangs using Klenow enzyme to facilitate religation and cloning.

After ethanol precipitation, the products were redissolved in 13 μl of ligation buffer, 1 μl T4-DNA ligase (15 units) and 1 μl T4 polynucleotide kinase were added, and the mixture was incubated at room temperature for 2 to 3 hours, or overnight at 16° C. Competent E. coli cells (in 40 μl of appropriate media) were transformed with 3 μl of ligation mixture and cultured in 80 μl of SOC medium. (See, e.g., Sambrook, supra, Appendix A, p. 2.) After incubation for one hour at 37° C., the E. coli mixture was plated on Luria Bertani (LB) agar (See, e.g., Sambrook, supra, Appendix A, p. 1) containing 2x Carb. The following day, several colonies were randomly picked from each plate and cultured in 150 μl of liquid LB/2x Carb medium placed in an individual well of an appropriate commercially-available sterile 96-well microtiter plate. The following day, 5 μl of each overnight culture was transferred into a non-sterile 96-well plate and, after dilution 1:10 with water, 5 μl from each sample was transferred into a PCR array.

For PCR amplification, 18 μl of concentrated PCR reaction mix (3.3x) containing 4 units of rTth DNA polymerase, a vector primer, and one or both of the gene specific primers used for the extension reaction were added to each well. Amplification was performed using the following conditions:

| | |
|---|---|
| Step 1 | 94° C. for 60 sec |
| Step 2 | 94° C. for 20 sec |
| Step 3 | 55° C. for 30 sec |
| Step 4 | 72° C. for 90 sec |
| Step 5 | Repeat steps 2 through 4 for an additional 29 cycles |
| Step 6 | 72° C. for 180 sec |
| Step 7 | 4° C. (and holding) |

Aliquots of the PCR reactions were run on agarose gels together with molecular weight markers. The sizes of the PCR products were compared to the original partial cDNAs, and appropriate clones were selected, ligated into plasmid, and sequenced.

In like manner, the nucleotide sequences of SEQ ID NO:18 through SEQ ID NO:34, are used to obtain 5′ regulatory sequences using the procedure above, oligonucleotides designed for 5′ extension, and an appropriate genomic library.

VI. Labeling and Use of Individual Hybridization Probes

Hybridization probes derived from SEQ ID NO:18 through SEQ ID NO:34, are employed to screen cDNAs, genomic DNAs, or mRNAs. Although the labeling of oligonucleotides, consisting of about 20 base pairs, is specifically described, essentially the same procedure is used with larger nucleotide fragments. Oligonucleotides are designed using state-of-the-art software such as OLIGO 4.06 (National Biosciences) and labeled by combining 50 pmol of each oligomer, 250 μCi of [γ-³²P] adenosine triphosphate (Amersham, Chicago, Ill.), and T4 polynucleotide kinase (DuPont NEN, Boston, Mass.). The labeled oligonucleotides are substantially purified using a Sephadex G-25 superfine resin column (Pharmacia & Upjohn, Kalamazoo, Mich.). An aliquot containing 10⁷ counts per minute of the labeled probe is used in a typical membrane-based hybridization analysis of human genomic DNA digested with one of the following endonucleases: Ase I, Bgl II, Eco RI, Pst I, Xba I, or Pvu II (DuPont NEN, Boston, Mass.).

The DNA from each digest is fractionated on a 0.7 percent agarose gel and transferred to nylon membranes (Nytran Plus, Schleicher & Schuell, Durham, N.H.). Hybridization is carried out for 16 hours at 40° C. To remove nonspecific signals, blots are sequentially washed at room temperature under increasingly stringent conditions up to 0.1×saline sodium citrate and 0.5% sodium dodecyl sulfate. After XOMAT AR film (Kodak, Rochester, N.Y.) is exposed to the blots to film for several hours, hybridization patterns are compared visually.

VII. Microarrays

A chemical coupling procedure and an ink jet device can be used to synthesize array elements on the surface of a substrate. (See, e.g., Baldeschweiler, supra.) An array analogous to a dot or slot blot may also be used to arrange and link elements to the surface of a substrate using or thermal, UV, mechanical, or chemical bonding procedures, or a vacuum system. A typical array may be produced by hand or using available methods and machines and contain any appropriate number of elements. After hybridization, nonhybridized probes are removed and a scanner used to determine the levels and patterns of fluorescence. The degree of complementarity and the relative abundance of each probe which hybridizes to an element on the microarray may be assessed through analysis of the scanned images.

In another alternative, full-length cDNAs or Expressed Sequence Tags (ESTs) comprise the elements of the microarray. Full-length cDNAs or ESTs corresponding to one of the nucleotide sequences of the present invention, or selected at random from a cDNA library relevent to the present invention, are arranged on an appropriate substrate, e.g., a glass slide. The cDNA is fixed to the slide using, e.g., U.V. cross-linking followed, by thermal and chemical and subsequent drying. (See, e.g., Schena, M. et al. (1995) Science 270:467-470; and Shalon, D. et al. (1996) Genome Res. 6:639-645.) Fluorescent probes are prepared and used for hybridization to the elements on the substrate. The substrate is analyzed by procedures described above.

Probe sequences for microarrays may be selected by screening a large number of clones from a variety of cDNA libraries in order to find sequences with conserved protein motifs common to genes coding for signal sequence containing polypeptides. In one embodiment, sequences identified from cDNA libraries, are analyzed to identify those gene sequences with conserved protein motifs using an appropriate analysis program, e.g., the Block 2 Bioanalysis Program (Incyte, Palo Alto, Calif.). This motif analysis program, based on sequence information contained in the Swiss-Prot Database and PROSITE, is a method of determining the function of uncharacterized proteins translated from genomic or cDNA sequences. (See, e.g., Bairoch, A. et al. (1997) Nucleic Acids Res. 25:217-221; and Attwood, T. K. et al. (1997) J. Chem. Inf. Comput. Sci. 37:417-424.) PROSITE may be used to identify functional or structural domains that cannot be detected using conserved motifs due to extreme sequence divergence. The method is based on weight matrices. Motifs identified by this method are then calibrated against the SWISS-PROT database in order to obtain a measure of the chance distribution of the matches.

In another embodiment, Hidden Markov models (HMMs) may be used to find shared motifs, specifically consensus sequences. (See, e.g., Pearson, W. R. and D. J. Lipman (1988) Proc. Natl. Acad. Sci. USA 85:2444-2448; and Smith, T. F. and M. S. Waterman (1981) J. Mol. Biol. 147:195-197.) HMMs were initially developed to examine speech recognition patterns, but are now being used in a biological context to analyze protein and nucleic acid sequences as well as to model protein structure. (See, e.g., Krogh, A. et al. (1994) J. Mol. Biol. 235:1501-1531; and Collin, M. et al. (1993) Protein Sci. 2:305-314.) HMMs have a formal probabilistic basis and use position-specific scores for amino acids or nucleotides. The algorithm continues to incorporate information from newly identified sequences to increase its motif analysis capabilities.

VIII. Complementary Polynucleotides

Sequences complementary to the MSP-encoding sequences, or any parts thereof, are used to detect, decrease, or inhibit expression of naturally occurring MSP. Although use of oligonucleotides comprising from about 15 to 30 base pairs is described, essentially the same procedure is used with smaller or with larger sequence fragments. Appropriate oligonucleotides are designed using Oligo 4.06 software and the coding sequence of MSP. To inhibit transcription, a complementary oligonucleotide is designed from the most unique 5′ sequence and used to prevent promoter binding to the coding sequence. To inhibit translation, a complementary oligonucleotide is designed to prevent ribosomal binding to the MSP-encoding transcript.

IX. Expression of MSP

Expression of MSP is accomplished by subcloning the cDNA into an appropriate vector and transforming the vector into host cells. This vector contains an appropriate promoter, e.g., β-galactosidase, upstream of the cloning site, operably associated with the cDNA of interest. (See, e.g., Sambrook, supra, pp. 404-433; and Rosenberg, M. et al. (1983) Methods Enzymol. 101:123-138.)

Induction of an isolated, transformed bacterial strain with isopropyl beta-D-thiogalactopyranoside (IPTG) using standard methods produces a fusion protein which consists of the first 8 residues of β-galactosidase, about 5 to 15 residues of linker, and the full length protein. The signal residues direct the secretion of MSP into bacterial growth media which can be used directly in the following assay for activity.

X. Demonstration of MSP Activity

Given the chemical and structural similarity between the MSP and other members of the membrane spanning protein families, MSP is identified as a new member of the membrane spanning proteins and is presumed to be involved in the regulation of cell growth.

To demonstrate that increased levels of MSP expression correlates with decreased cell motility and increased cell proliferation, expression vectors encoding MSP are electroporated into highly motile cell lines, such as U-937 (ATCC CRL 1593), HEL 92.1.7 (ATCC TIB 180) and MAC10, and the motility of the electroporated and control cells are compared. Methods for the design and construction of an expression vector capable of expressing MSP in the desired mammalian cell line(s) chosen are well known to the art. Assays for examining the motility of cells in culture are known to the art (cf Miyake, M. et al. (1991) J. Exp. Med. 174:1347-1354 and Ikeyama, S. et al. (1993) J. Exp. Med. 177:1231-1237). Increasing the level of MSP in highly motile cell lines by transfection with an MSP expression vector inhibits or reduces the motility of these cell lines, and the amount of this inhibition is proportional top the activity of MSP in the assay.

XI. Production of MSP Specific Antibodies

MSP substantially purified using PAGE electrophoresis (see, e.g., Harrington, M. G. (1990) Methods Enzymol. 182:488-495), or other purification techniques, is used to immunize rabbits and to produce antibodies using standard protocols. The MSP amino acid sequence is analyzed using DNASTAR software (DNASTAR Inc) to determine regions of high immunogenicity, and a corresponding oligopeptide is synthesized and used to raise antibodies by means known to those of skill in the art. Methods for selection of appropriate epitopes, such as those near the C-terminus or in hydrophilic regions are well described in the art. (See, e.g., Ausubel et al. supra, ch. 11.)

Typically, the oligopeptides are 15 residues in length, and are synthesized using an Applied Biosystems Peptide Synthesizer Model 431A using fmoc-chemistry and coupled to KLH (Sigma, St. Louis, Mo.) by reaction with N-maleimidobenzoyl-N-hydroxysuccinimide ester (MBS) to increase immunogenicity. (See, e.g., Ausubel et al. supra.) Rabbits are immunized with the oligopeptide-KLH complex in complete Freund's adjuvant. Resulting antisera are tested for antipeptide activity, for example, by binding the peptide to plastic, blocking with 1% BSA, reacting with rabbit antisera, washing, and reacting with radio-iodinated goat anti-rabbit IgG.

XII. Purification of Naturally Occurring MSP Using Specific Antibodies

Naturally occurring or recombinant MSP is substantially purified by immunoaffinity chromatography using antibodies specific for MSP. An immunoaffinity column is constructed by covalently coupling anti-MSP antibody to an activated chromatographic resin, such as CNBr-activated Sepharose (Pharmacia & Upjohn). After the coupling, the resin is blocked and washed according to the manufacturer's instructions.

Media containing MSP are passed over the immunoaffinity column, and the column is washed under conditions that allow the preferential absorbance of MSP (e.g., high ionic strength buffers in the presence of detergent). The column is eluted under conditions that disrupt antibody/MSP binding (e.g., a buffer of pH 2 to pH 3, or a high concentration of a chaotrope, such as urea or thiocyanate ion), and MSP is collected.

XIII. Identification of Molecules Which Interact with MSP

MSP, or biologically active fragments thereof, are labeled with ¹²⁵I Bolton-Hunter reagent. (See, e.g., Bolton, A. E. and W. M. Hunter (1973) Biochem. J. 133:529-539.) Candidate molecules previously arrayed in the wells of a multi-well plate are incubated with the labeled MSP, washed, and any wells with labeled MSP complex are assayed. Data obtained using different concentrations of MSP are used to calculate values for the number, affinity, and association of MSP with the candidate molecules.

Various modifications and variations of the described methods and systems of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

What is claimed is:

1. An isolated polypeptide selected from the group consisting of:
a) a polypeptide comprising an amino acid sequence selected from the group consisting of SEQ ID NO:1-17,
b) a naturally occurring polypeptide comprising an amino acid sequence at least 90% identical to an amino acid sequence selected from the group consisting of SEQ ID NO:1-17,
c) a biologically active fragment of a polypeptide having an amino acid sequence selected from the group consisting of SEQ ID NO:1-17, and
d) an immunogenic fragment of a polypeptide having an amino acid sequence selected from the group consisting of SEQ ID NO:1-17.

2. An isolated polypeptide of claim 1 selected from the group consisting of SEQ ID NO:1-17.

3. An isolated polynucleotide encoding a polypeptide of claim 1.

4. An isolated polynucleotide encoding a polypeptide of claim 2.

5. An isolated polynucleotide of claim 4 selected from the group consisting of SEQ ID NO:18-34.

6. A recombinant polynucleotide comprising a promoter sequence operably linked to a polynucleotide of claim 3.

7. A cell transformed with a recombinant polynucleotide of claim 6.

8. A transgenic organism comprising a recombinant polynucleotide of claim 6.

9. A method for producing a polypeptide of claim 1, the method comprising:
a) culturing a cell under conditions suitable for expression of the polypeptide, wherein said cell is transformed with a recombinant polynucleotide, and said recombinant polynucleotide comprises a promoter sequence operably linked to a polynucleotide encoding the polypeptide of claim 1, and
b) recovering the polypeptide so expressed.

10. An isolated antibody which specifically binds to a polypeptide of claim 1.

11. An isolated polynucleotide selected from the group consisting of:
a) a polynucleotide comprising a polynucleotide sequence selected from the group consisting of SEQ ID NO:18-34,
b) a naturally occurring polynucleotide comprising a polynucleotide sequence at least 90% identical to a polynucleotide sequence selected from the group consisting of SEQ ID NO:18-34,
c) a polynucleotide complementary to the polynucleotide of a),
d) a polynucleotide complementary to the polynucleotide of b), and
e) an RNA equivalent of a)-d).

12. An isolated polynucleotide comprising at least 60 contiguous nucleotides of a polynucleotide of claim 11.

13. A method for detecting a target polynucleotide in a sample, said target polynucleotide having a sequence of a polynucleotide of claim 11, the method comprising:
a) hybridizing the sample with a probe comprising at least 20 contiguous nucleotides comprising a sequence complementary to said target polynucleotide in the sample, and which probe specifically hybridizes to said target polynucleotide, under conditions whereby a hybridization complex is formed between said probe and said target polynucleotide or fragments thereof, and
b) detecting the presence or absence of said hybridization complex, and, optionally, if present, the amount thereof.

14. A method of claim 13, wherein the probe comprises at least 60 contiguous nucleotides.

15. A method for detecting a target polynucleotide in a sample, said target polynucleotide having a sequence of a polynucleotide of claim 11, the method comprising:
a) amplifying said target polynucleotide or fragment thereof using polymerase chain reaction amplification, and
b) detecting the presence or absence of said amplified target polynucleotide or fragment thereof, and, optionally, if present, the amount thereof.

16. A composition comprising a polypeptide of claim 1 and a pharmaceutically acceptable excipient.

17. A composition of claim 16, wherein the polypeptide has an amino acid sequence selected from the group consisting of SEQ ID NO:1-17.

18. A method for treating a disease or condition associated with decreased expression of functional MSP, comprising administering to a patient in need of such treatment the composition of claim 16.

19. A method for screening a compound for effectiveness as an agonist of a polypeptide of claim 1, the method comprising:
a) exposing a sample comprising a polypeptide of claim 1 to a compound, and
b) detecting agonist activity in the sample.

20. A composition comprising an agonist compound identified by a method of claim 19 and a pharmaceutically acceptable excipient.

21. A method for treating a disease or condition associated with decreased expression of functional MSP, comprising administering to a patient in need of such treatment a composition of claim 20.

22. A method for screening a compound for effectiveness as an antagonist of a polypeptide of claim 1, the method comprising:
a) exposing a sample comprising a polypeptide of claim 1 to a compound, and
b) detecting antagonist activity in the sample.

23. A composition comprising an antagonist compound identified by a method of claim 22 and a pharmaceutically acceptable excipient.

24. A method for treating a disease or condition associated with overexpression of functional MSP, comprising administering to a patient in need of such treatment a composition of claim 23.

25. A method of screening for a compound that specifically binds to the polypeptide of claim 1, said method comprising the steps of:
a) combining the polypeptide of claim 1 with at least one test compound under suitable conditions, and
b) detecting binding of the polypeptide of claim 1 to the test compound, thereby identifying a compound that specifically binds to the polypeptide of claim 1.

26. A method of screening for a compound that modulates the activity of the polypeptide of claim 1, said method comprising:
a) combining the polypeptide of claim 1 with at least one test compound under conditions permissive for the activity of the polypeptide of claim 1,
b) assessing the activity of the polypeptide of claim 1 in the presence of the test compound, and
c) comparing the activity of the polypeptide of claim 1 in the presence of the test compound with the activity of the polypeptide of claim 1 in the absence of the test compound, wherein a change in the activity of the polypeptide of claim 1 in the presence of the test compound is indicative of a compound that modulates the activity of the polypeptide of claim 1.

27. A method for screening a compound for effectiveness in altering expression of a target polynucleotide, wherein said target polynucleotide comprises a sequence of claim 5, the method comprising:
a) exposing a sample comprising the target polynucleotide to a compound, under conditions suitable for the expression of the target polynucleotide,
b) detecting altered expression of the target polynucleotide, and
c) comparing the expression of the target polynucleotide in the presence of varying amounts of the compound and in the absence of the compound.

28. A method for assessing toxicity of a test compound, said method comprising:
a) treating a biological sample containing nucleic acids with the test compound;
b) hybridizing the nucleic acids of the treated biological sample with a probe comprising at least 20 contiguous nucleotides of a polynucleotide of claim 11 under conditions whereby a specific hybridization complex is formed between said probe and a target polynucleotide in the biological sample, said target polynucleotide comprising a polynucleotide sequence of a polynucleotide of claim 11 or fragment thereof;
c) quantifying the amount of hybridization complex; and
d) comparing the amount of hybridization complex in the treated biological sample with the amount of hybridization complex in an untreated biological sample, wherein a difference in the amount of hybridization complex in the treated biological sample is indicative of toxicity of the test compound.

29. A diagnostic test for a condition or disease associated with the expression of MSP in a biological sample comprising the steps of:
a) combining the biological sample with an antibody of claim 10, under conditions suitable for the antibody to bind the polypeptide and form an antibody:polypeptide complex; and
b) detecting the complex, wherein the presence of the complex correlates with the presence of the polypeptide in the biological sample.

30. The antibody of claim 10, wherein the antibody is:
a) a chimeric antibody,
b) a single chain antibody,
c) a Fab fragment,
d) a F(ab′)₂ fragment, or
e) a humanized antibody.

31. A composition comprising an antibody of claim 10 and an acceptable excipient.

32. A method of diagnosing a condition or disease associated with the expression of MSP in a subject, comprising administering to said subject an effective amount of the composition of claim 31.

33. A composition of claim 31, wherein the antibody is labeled.

34. A method of diagnosing a condition or disease associated with the expression of MSP in a subject, comprising administering to said subject an effective amount of the composition of claim 33.

35. A method of preparing a polyclonal antibody with the specificity of the antibody of claim 10 comprising:
a) immunizing an animal with a polypeptide having an amino acid sequence selected from the group consisting of SEQ ID NO:1-17, or an immunogenic fragment thereof, under conditions to elicit an antibody response;
b) isolating antibodies from said animal; and
c) screening the isolated antibodies with the polypeptide, thereby identifying a polyclonal antibody which binds specifically to a polypeptide having an amino acid sequence selected from the group consisting of SEQ ID NO:1-17.

36. An antibody produced by a method of claim 35.

37. A composition comprising the antibody of claim 36 and a suitable carrier.

38. A method of making a monoclonal antibody with the specificity of the antibody of claim 10 comprising:
a) immunizing an animal with a polypeptide having an amino acid sequence selected from the group consisting of SEQ ID NO:1-17, or an immunogenic fragment thereof, under conditions to elicit an antibody response;
b) isolating antibody producing cells from the animal;
c) fusing the antibody producing cells with immortalized cells to form monoclonal antibody-producing hybridoma cells;
d) culturing the hybridoma cells; and
e) isolating from the culture monoclonal antibody which binds specifically to a polypeptide having an amino acid sequence selected from the group consisting of SEQ ID NO:1-17.

39. A monoclonal antibody produced by a method of claim 38.

40. A composition comprising the antibody of claim 39 and a suitable carrier.

41. The antibody of claim 10, wherein the antibody is produced by screening a Fab expression library.

42. The antibody of claim 10, wherein the antibody is produced by screening a recombinant immunoglobulin library.

43. A method for detecting a polypeptide having an amino acid sequence selected from the group consisting of SEQ ID NO:1-17 in a sample, comprising the steps of:
a) incubating the antibody of claim 10 with a sample under conditions to allow specific binding of the antibody and the polypeptide; and
b) detecting specific binding, wherein specific binding indicates the presence of a polypeptide having an amino acid sequence selected from the group consisting of SEQ ID NO:1-17 in the sample.

44. A method of purifying a polypeptide having an amino acid sequence selected from the group consisting of SEQ ID NO:1-17 from a sample, the method comprising:
a) incubating the antibody of claim 10 with a sample under conditions to allow specific binding of the antibody and the polypeptide; and
b) separating the antibody from the sample and obtaining the purified polypeptide having an amino acid sequence selected from the group consisting of SEQ ID NO:1-17.

45. A polypeptide of claim 1, comprising the amino acid sequence of SEQ ID NO:1.

46. A polypeptide of claim 1, comprising the amino acid sequence of SEQ ID NO:2.

47. A polypeptide of claim 1, comprising the amino acid sequence of SEQ ID NO:3.

48. A polypeptide of claim 1, comprising the amino acid sequence of SEQ ID NO:4.

49. A polypeptide of claim 1, comprising the amino acid sequence of SEQ ID NO:5.

50. A polypeptide of claim 1, comprising the amino acid sequence of SEQ ID NO:6.

51. A polypeptide of claim 1, comprising the amino acid sequence of SEQ ID NO:7.

52. A polypeptide of claim 1, comprising the amino acid sequence of SEQ ID NO:8.

53. A polypeptide of claim 1, comprising the amino acid sequence of SEQ ID NO:9.

54. A polypeptide of claim 1, comprising the amino acid sequence of SEQ ID NO:10.

55. A polypeptide of claim 1, comprising the amino acid sequence of SEQ ID NO:11.

56. A polypeptide of claim 1, comprising the amino acid sequence of SEQ ID NO:12.

57. A polypeptide of claim 1, comprising the amino acid sequence of SEQ ID NO:13.

58. A polypeptide of claim 1, comprising the amino acid sequence of SEQ ID NO:14.

59. A polypeptide of claim 1, comprising the amino acid sequence of SEQ ID NO:15.

60. A polypeptide of claim 1, comprising the amino acid sequence of SEQ ID NO:16.

61. A polypeptide of claim 1, comprising the amino acid sequence of SEQ ID NO:17.

62. A polynucleotide of claim 11, comprising the polynucleotide sequence of SEQ ID NO:18.

63. A polynucleotide of claim 11, comprising the polynucleotide sequence of SEQ ID NO:19.

64. A polynucleotide of claim 11, comprising the polynucleotide sequence of SEQ ID NO:20.

65. A polynucleotide of claim 11, comprising the polynucleotide sequence of SEQ ID NO:21.

66. A polynucleotide of claim 11, comprising the polynucleotide sequence of SEQ ID NO:22.

67. A polynucleotide of claim 11, comprising the polynucleotide sequence of SEQ ID NO:23.

68. A polynucleotide of claim 11, comprising the polynucleotide sequence of SEQ ID NO:24.

69. A polynucleotide of claim 11, comprising the polynucleotide sequence of SEQ ID NO:25.

70. A polynucleotide of claim 11, comprising the polynucleotide sequence of SEQ ID NO:26.

71. A polynucleotide of claim 11, comprising the polynucleotide sequence of SEQ ID NO:27.

72. A polynucleotide of claim 11, comprising the polynucleotide sequence of SEQ ID NO:28.

73. A polynucleotide of claim 11, comprising the polynucleotide sequence of SEQ ID NO:29.

74. A polynucleotide of claim 11, comprising the polynucleotide sequence of SEQ ID NO:30.

75. A polynucleotide of claim 11, comprising the polynucleotide sequence of SEQ ID NO:31.

76. A polynucleotide of claim 11, comprising the polynucleotide sequence of SEQ ID NO:32.

77. A polynucleotide of claim 11, comprising the polynucleotide sequence of SEQ ID NO:33.

78. A polynucleotide of claim 11, comprising the polynucleotide sequence of SEQ ID NO:34.
